(19) United States
(12) Patent Application Publication
(10) Pub. No.: US 2024/0189060 A1
(43) Pub. Date: Jun. 13, 2024

(54) Systems, Methods and Apparatus for Points-Based Registration of a Medical Hologram to a Surgical Site in Holographic Medical Augmented Reality

(71) Applicant: apoQlar GmbH, Hamburg (DE)

(72) Inventors: Erke Can Tellal, Hamburg (DE); Sirko Pelzl, Hamburg (DE); Kevin Peivareh, Hamburg (DE); Marc-Angelo Bisotti, Hamburg (DE); Roman Kupalov, Hamburg (DE)

(73) Assignee: apoQlar GmbH, Hamburg (DE)

(21) Appl. No.: 18/076,372

(22) Filed: Dec. 6, 2022

(51) Int. Cl. A61B 90/00

(52) U.S. Cl. CPC A61B 90/36; A61B 2090/365

(57) ABSTRACT

Systems, methods and apparatus are provided through which in some implementations registration points are created on a medical hologram, the registration points are placed on a surgical site and the medical hologram is registered on the surgical site.

### FIELD

This disclosure relates generally to medical augmented reality, and more particularly to points-based superimposition of a patient specific anatomy hologram and/or virtual surgical planning tools to a surgical site in holographic medical augmented reality.

### BACKGROUND

During a conventional image-guided surgery, operators must constantly switch their perspective between a surgical site and the 2D navigation display. A surgical site can be a patient situs, a phantom situs or a planning situs. Moreover, the operators must spend a great deal of mental effort for mapping the information displayed on the 2D display to the actual surgical site. On one hand this mental discrepancy can result in increased risk to patient safety. On the other hand, this mental discrepancy prevents less or unexperienced operators to perform surgeries as efficient as more experienced ones. Augmented reality offers a great opportunity for overcoming this problem; because augmented reality allows not only to superimpose virtual objects on the real world, but also establishes relations between virtual objects and the real world by means of spatial mapping. However, the biggest challenge of the whole workflow is the accurate superimposition of virtual data on the surgical site. There are already various methods to overcome, however all the various methods have limitations in their own.

The manual registration is a slow and inaccurate method since it requires user to transform and translate the medical hologram at the same time. It has also low accuracy due to the perspective change when looking at the medical hologram from different angles.

### BRIEF DESCRIPTION

Fully automatic registration makes use of Artificial Intelligence models that are trained for recognizing landmarks from specific organ/part of the body. However, when the patient is draped on the operating table, landmarks are also covered under the sheet and are not exposed to depth sensors of the augmented reality devices.

Marker based registration requires attaching rigid preoperative reflective markers to the patient before the acquisition of medical images. These markers are then used as spatial anchors for the mixed reality headset. However, this approach requires a lengthy preparation time and only applicable to medical images that are taken exclusively.

The main aim benefit of the systems, methods and apparatus disclosed herein to provide a flexible, fast, and accurate solution which can be easily integrated to existing surgical workflows.

The above-mentioned shortcomings, disadvantages and problems are addressed herein, which will be understood by reading and studying the following specification.

In one aspect, a method creates registration points on a patient specific anatomy hologram, places the same registration points on a surgical site and automatically superimposes the medical hologram on the surgical site.

Apparatus, systems, and methods of varying scope are described herein. In addition to the aspects and advantages described in this summary, further aspects and advantages will become apparent by reference to the drawings and by reading the detailed description that follows.

### BRIEF DESCRIPTION OF THE DRAWINGS

FIG. 1 is a system of points-based registration of medical images to a patient in holographic medical augmented reality, according to an implementation.

FIG. 2 is a selector of a medical hologram of a patient, according to an implementation.

FIG. 3 is a graphical user interface of a selected medical hologram of a patient, according to an implementation.

FIG. 4 is a registration selector providing the selection of the selected medical hologram of a patient, according to an implementation.

FIG. 5 is a graphical user interface showing a placement tool, according to an implementation.

FIG. 6 is a manual creator of registration points on the medical hologram, according to an implementation.

FIG. 7 is an automatic creator of registration points on the medical hologram, according to an implementation.

FIG. 8 is a graphic user interface of creating points on the medical hologram, according to an implementation.

FIG. 9 is a graphical user interface showing the next button for point placement on a surgical site, according to an implementation.

FIG. 10 is a block diagram of a placer of registration points on a surgical site, according to an implementation.

FIG. 11 is a graphical user interface of placing points on the surgical site according to an implementation that is performed by FIG. 10.

FIG. 12 is a registrar of the medical hologram on a surgical site, according to an implementation.

FIG. 13 is a graphical user interface of a next button for registration of the medical hologram, according to an implementation that is performed by FIG. 12.

FIG. 14 is a graphical user interface of rotation and translation of the medical hologram to the patient's position, according to an implementation that is performed by FIG. 12.

FIG. 15 is a flowchart of a method of points-based registration of a medical image to a patient in holographic medical augmented reality, according to an implementation.

FIG. 16 is a flowchart of a method to select a medical hologram of a patient, according to an implementation.

FIG. 17 is a flow chart of a method of selecting registration, according to an implementation.

FIG. 18 is a flow chart of a method of creating registration points on the medical hologram, according to an implementation

FIG. 19 is a flow chart of a method of automatically creating registration points on the medical hologram 1900, according to an implementation.

FIG. 20 is a flow chart of a method of placing registration points on a surgical site, according to an implementation.

FIG. 21 is a method of registration of the medical hologram on the surgical site, according to an implementation.

FIG. 22 is a flowchart of a method of points-based registration of a medical image to a patient in holographic medical augmented reality, according to an implementation.

FIG. 23 is a flow chart of a method of editing registration points, according to an implementation.

FIG. 24 is a flow chart of a method of editing registration points, according to an implementation.

FIG. 25 is a graphical user interface showing the editing of registration points on a surgical site, according to an implementation

FIG. 26 is a flowchart of a method of creating registration points on a medical hologram, according to an implementation.

FIG. 27 is a UML diagram of a placement feature class in point based registration of a medical image of a patient in holographic medical augmented reality, according to an implementation.

FIG. 28 is a UML diagram of a placement engine class in point based registration of a medical image of a patient in holographic medical augmented reality according to an implementation.

FIG. 29 is a UML diagram of the abstract placement class in point based registration of a medical image of a patient in holographic medical augmented reality, according to an implementation.

FIG. 30 is a UML diagram of a monobehavior class in point based registration of a medical image of a patient in holographic medical augmented reality, according to an implementation.

FIG. 31 is a diagram describing finger tracking, according to an implementation.

FIG. 32 is a block diagram of a holographic medical augmented reality control computer in which different implementations can be practiced.

FIG. 33 is a block diagram of a data acquisition circuit of the holographic medical augmented reality control computer, according to an implementation.

FIG. 34 is a block diagram of a hardware and operating environment in which different implementations can be practiced.

FIG. 35 is a block diagram of a holographic medical augmented reality control mobile device, according to an implementation.

### DETAILED DESCRIPTION

In the following detailed description, reference is made to the accompanying drawings that form a part hereof, and in which is shown by way of illustration specific implementations which may be practiced. These implementations are described in sufficient detail to enable those skilled in the art to practice the implementations, and it is to be understood that other implementations may be utilized, and that logical, mechanical, electrical and other changes may be made without departing from the scope of the implementations. The following detailed description is, therefore, not to be taken in a limiting sense.

The detailed description is divided into five sections. In the first section, a system level overview is described. In the second section, apparatus of implementations are described. In the third section, implementations of methods are described. In the fourth section, implementations of classes instantiated in the system 100, apparatus 200, 400, 600, 700, 1000 and 1200 and methods 1500-2600 are described. In the fifth section, implementations of hardware, and the operating environment in conjunction with which implementations may be practiced are described. Finally, in the sixth section, a conclusion of the detailed description is provided.

### System Level Overview

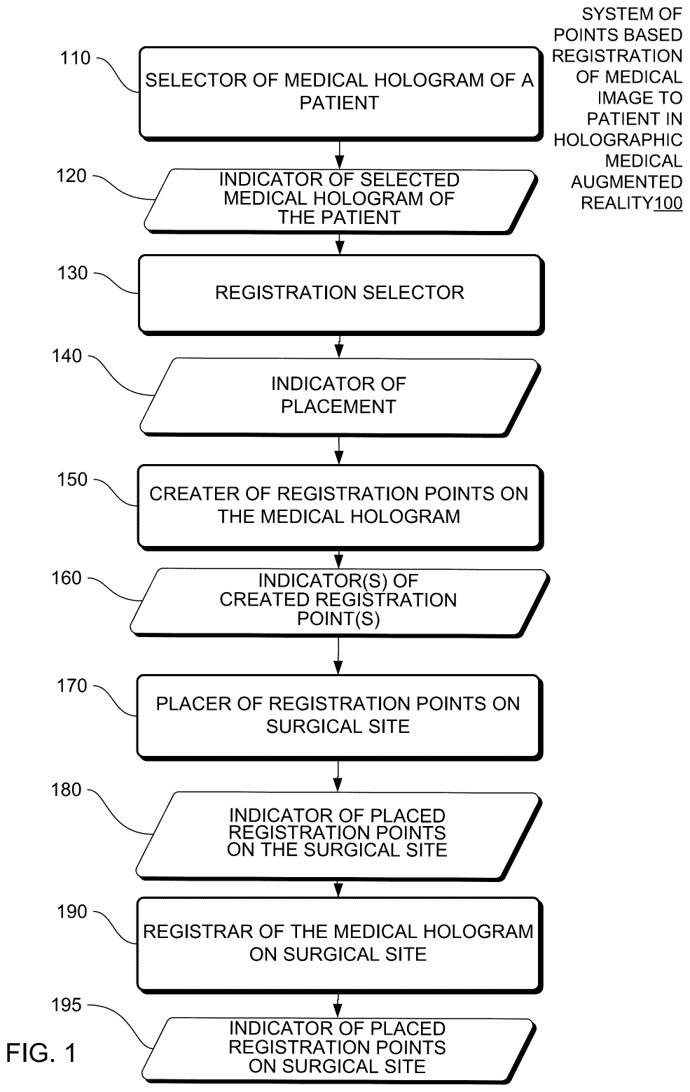

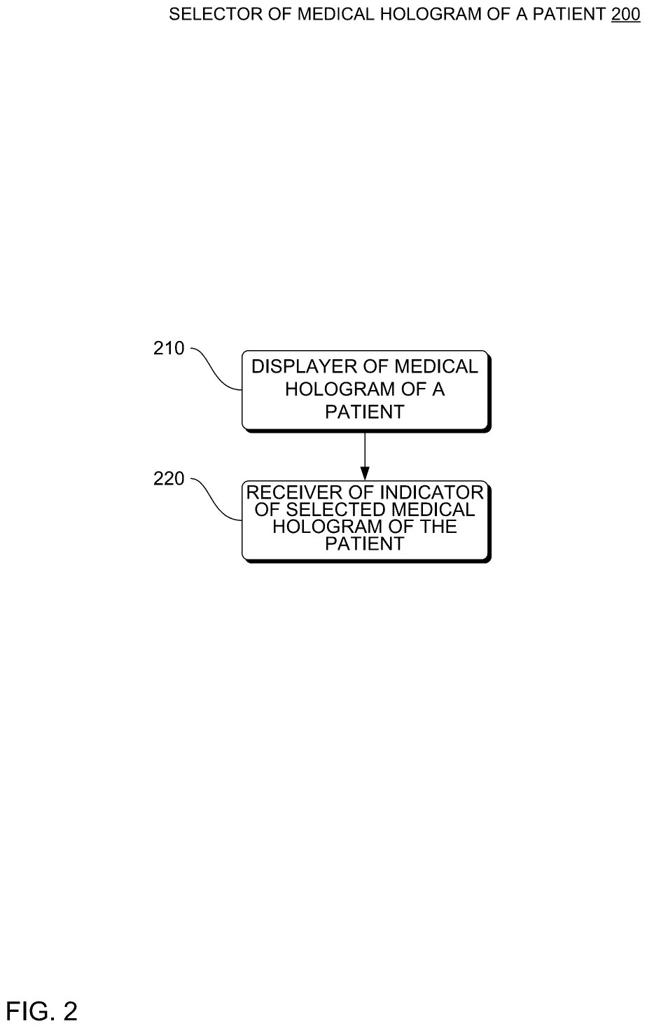

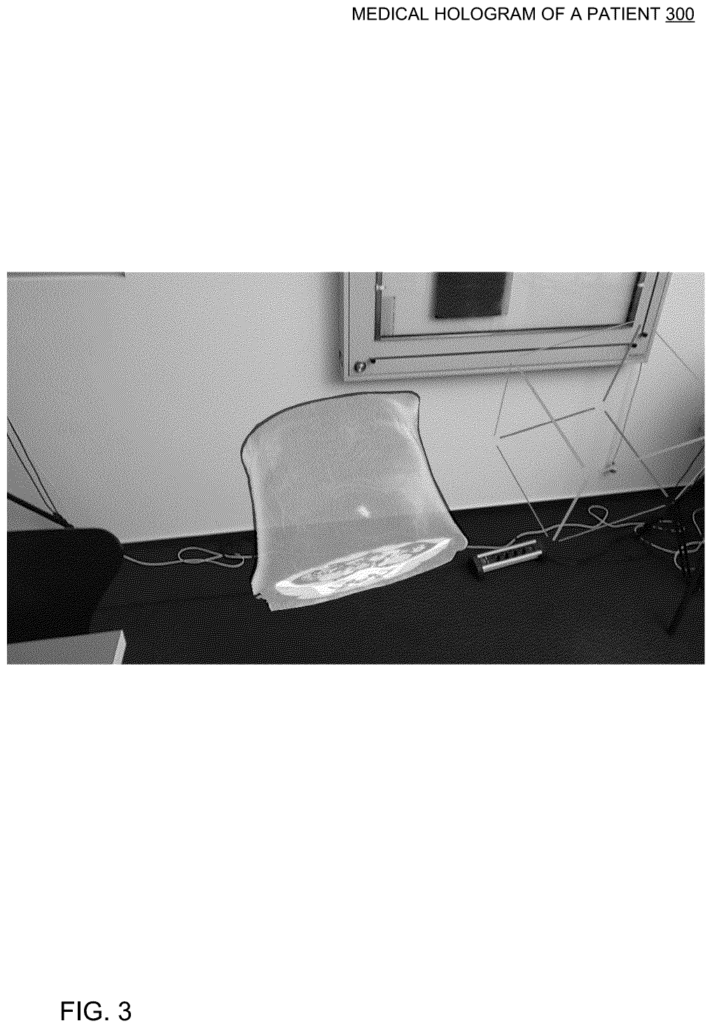

FIG. 1 is a system 100 of points-based registration of medical images to a patient in holographic medical augmented reality, according to an implementation. System 100 includes a selector 110 of a medical hologram of a patient created from a medical image. One example of the medical hologram of a patient is medical hologram of a patient 300 in FIG. 3. System 100 and apparatus 200, 400, 600, 700, 1000 and 1200 and methods 1500-2600 are operable using any kind of 3D medical image, such as computed tomography, magnetic resonance imaging, stereolithography or any other medical image data format. Any anatomical part of a patient, such as a knee, head, neck, abdomen can be the anatomical subject of the system 100, apparatus 200, 400, 600, 700, 1000 and 1200 and methods 1500-2600. The selector 110 generates an indicator 120 of the selected medical hologram of a patient. The selector 110 is described in greater detail in FIG. 2.

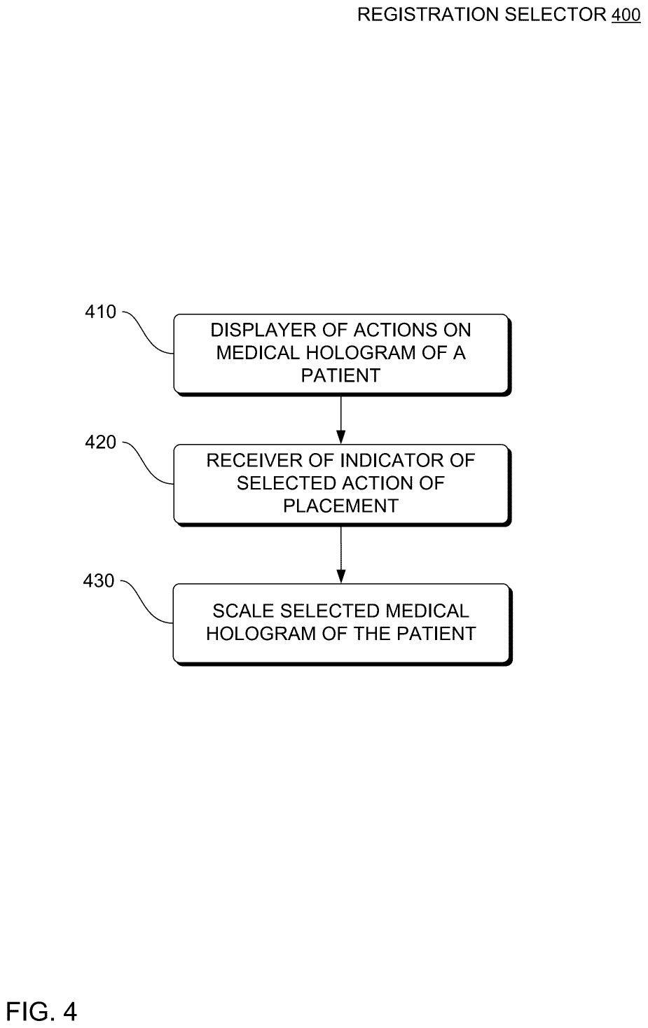

Once a medical hologram of a patient is selected, the user can start the point-based registration process by selecting “placement” from the object toolbox menu, accordingly, in some implementations, the selector 110 is operably coupled to a registration selector 130 that receives the indicator 120 and generates an indicator 140 of placement. The registration selector 130 is described in greater detail in FIG. 4. The registration elector 130 is operably coupled to a creator 150 of registration points on the medical hologram.

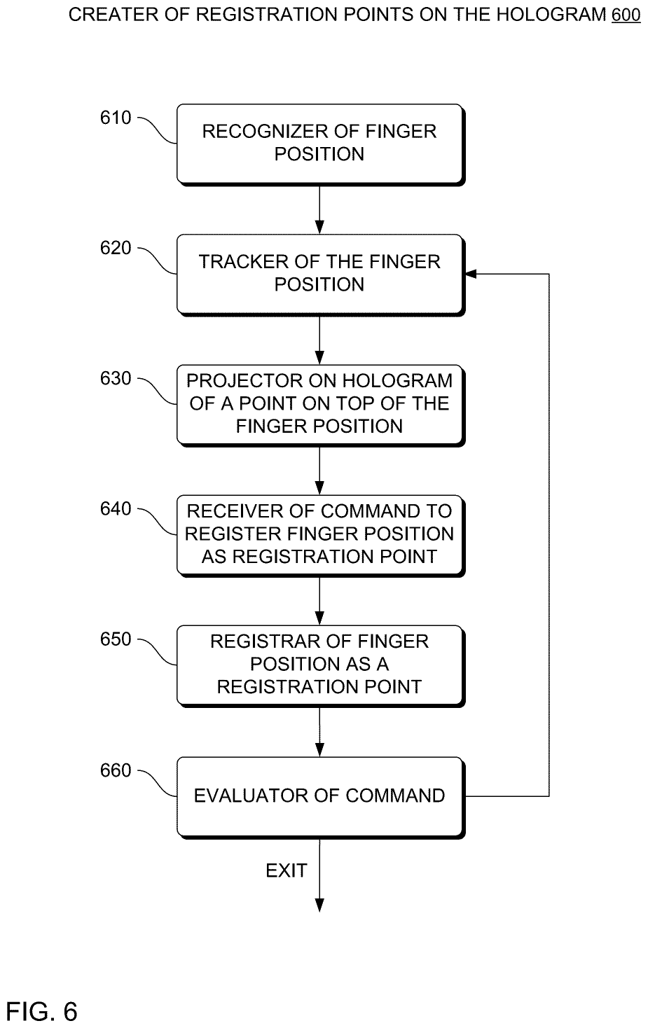

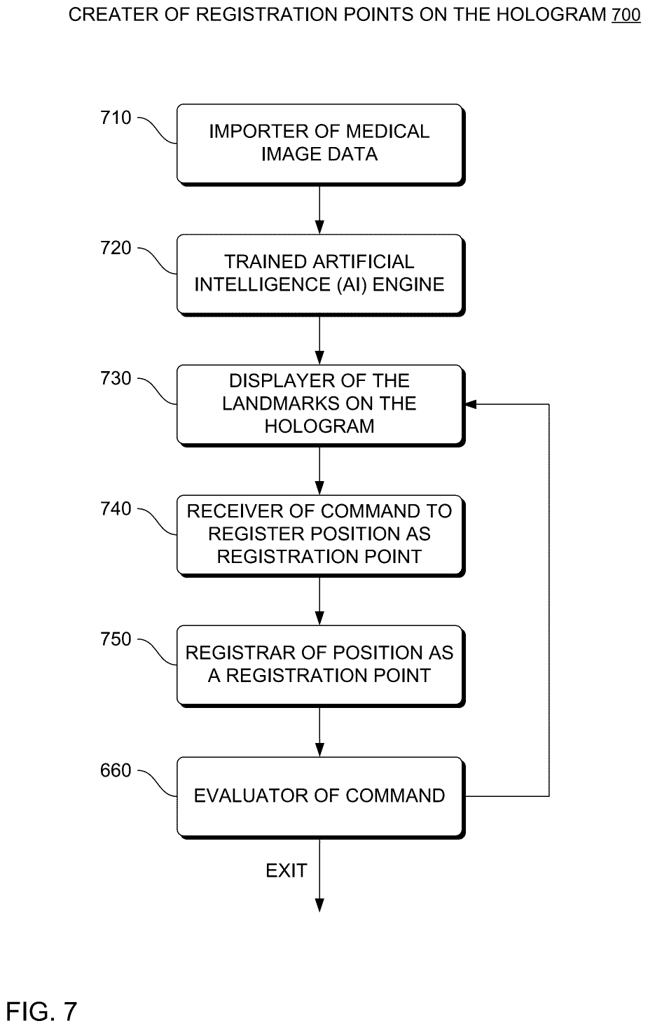

The creator 150 generates an indicator of created registration points 160. The creator 150 is described in greater detail in FIG. 6 and FIG. 7. In some implementations, the user can provide the speech command “Undo” to remove the last created registration point. The creator 150 is operably coupled to a placer 170 of registration points on the surgical site.

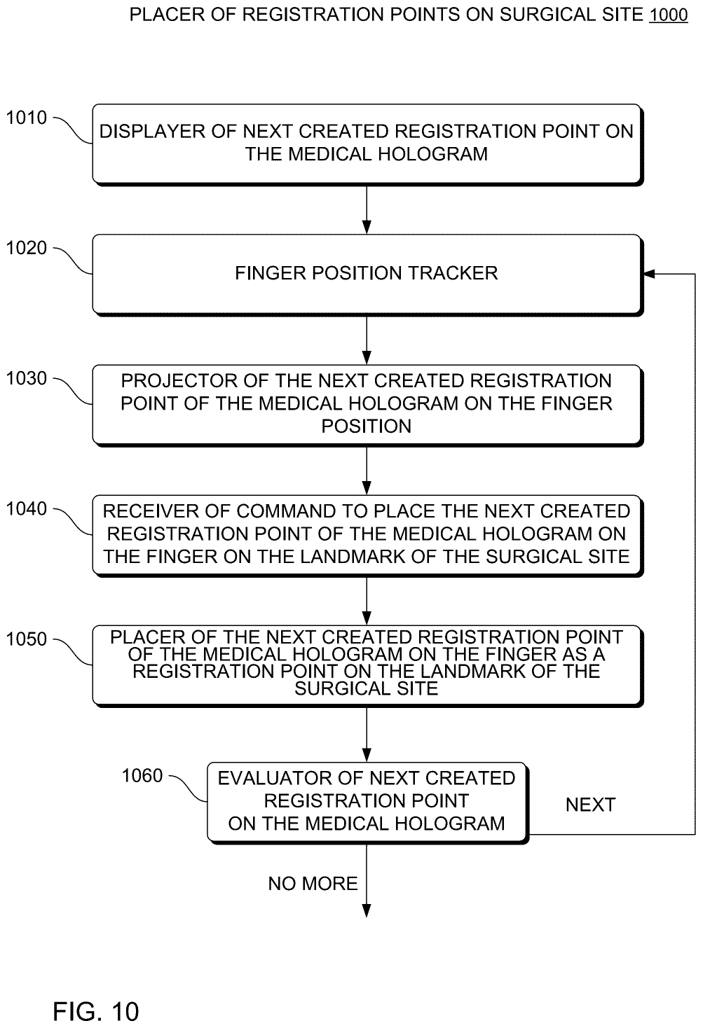

The placer 170 generates an indicator 180 of placed registration points on the surgical site. The placer 170 is described in greater detail in FIG. 10.

In some implementations, 3, 4, 5 or more points can be placed, depending on the availability of patient's anatomical landmarks for use. It is completely up to the user to decide how many points they would like to use. In some implementations, the points can be moved so that the points have the correct position. Points that are previously created on the medical hologram are displayed.

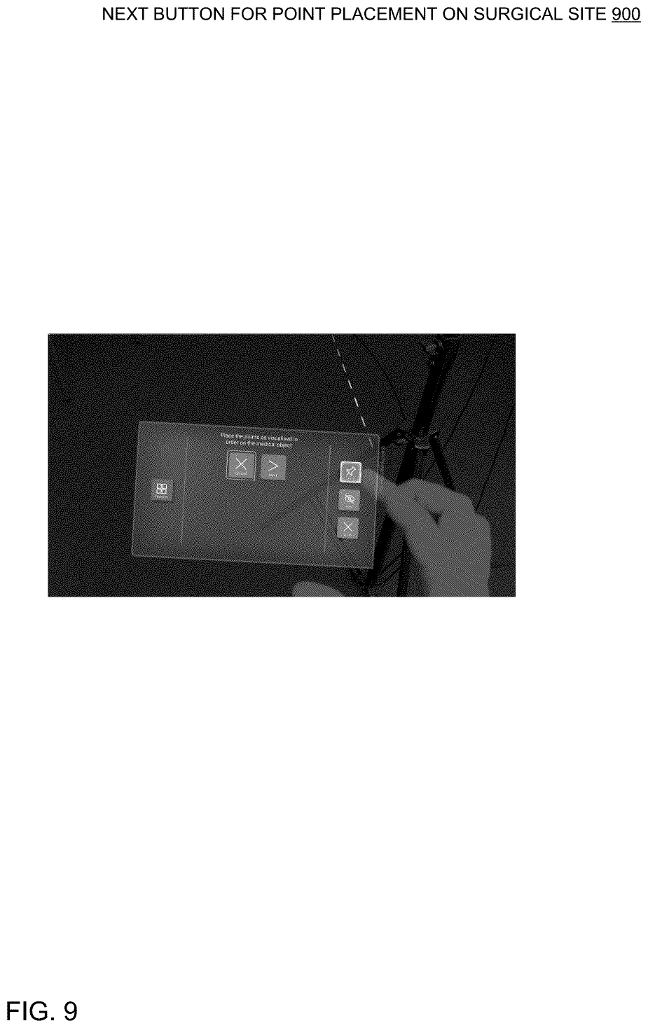

When the user is satisfied with the position and quantity of registration points, the user can click next in the object toolbox menu, as shown in FIG. 9.

The placer 170 is operably coupled to a registrar 190 of the medical hologram on the surgical site.

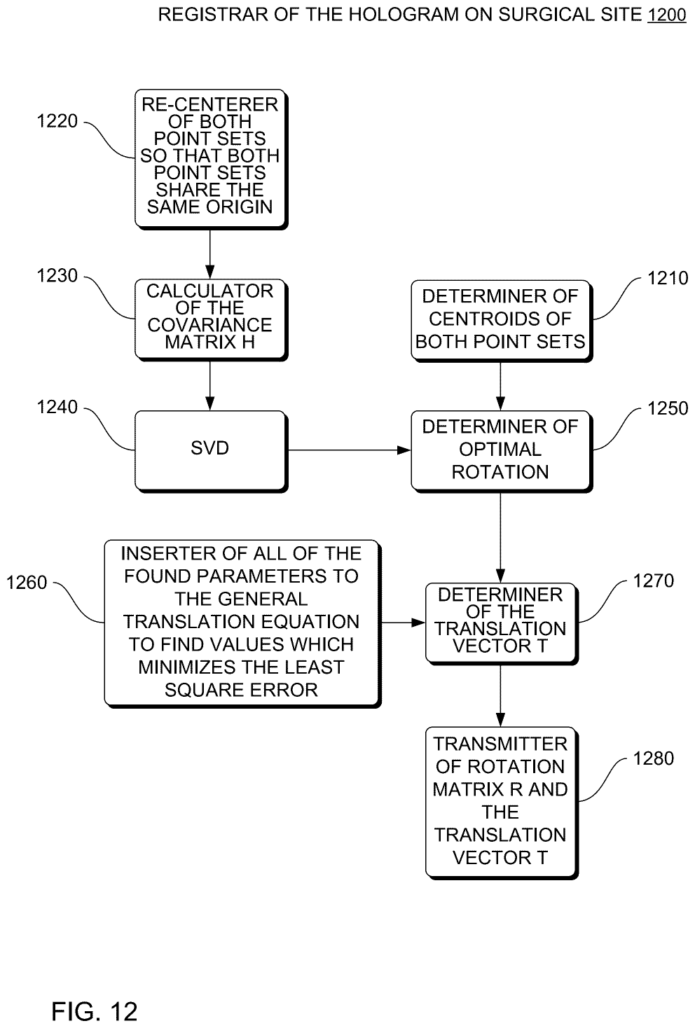

The registrar 190 generates an indicator of place registration points on the surgical site 195. The points must be registered in same order as it was done during the previous step for creating the points on the medical hologram, thus in some implementations, there is a color coding or numbering. The color of the point projected on the finger, matches the color of the point from the medical hologram that needs to be placed on the surgical site. The registrar 190 is described in greater detail in FIG. 12.

### Apparatus Implementations

In the previous section, a system level overview of the operation of an implementation was described. In this section, the particular apparatus of such an implementation are described by reference to a series of diagrams.

FIG. 2 is a selector of a medical hologram of a patient 200, according to an implementation. The selector 200 is one implementation of the selector 110 in FIG. 1. The selector 200 includes a displayer of a medical hologram of a patient 210. That is operably coupled to a receiver of an indicator of a selected medical hologram of a patient 220. The indicator of the selected medical hologram of a patient in FIG. 2 is the same as the indicator 120 of selected medical hologram of a patient in FIG. 1.

FIG. 3 is a graphical user interface of a selected medical hologram of a patient 300, according to an implementation.

FIG. 4 is a registration selector 400 providing the selection of the placement for selected medical hologram of a patient, according to an implementation. FIG. 4 includes a registration selector 400, according to an implementation. The registration selector 400 is one implementation of the registration selector 130 in FIG. 1. The registration selector 400 includes a displayer of actions that can be performed on a medical hologram of a patient 410 that is operably coupled to a receiver 420 of an indication of a selected action of placement. The receiver 420 is operably coupled to a scaler of the selected medical hologram of a patient 430. In some implementations, the medical hologram of a patient is scaled to 1:1 size, to make sure that registration happens with the correct geometric dimensions. The registration selector 400 generates the indicator 140 of placement in FIG. 1.

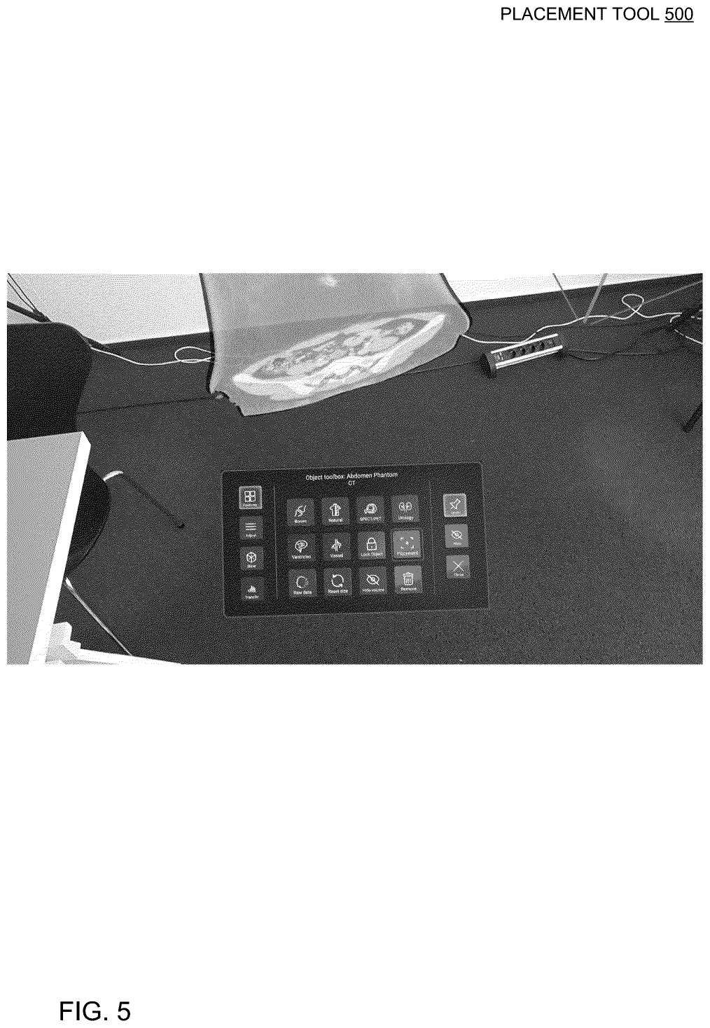

FIG. 5 is a graphical user interface showing a placement tool 500, according to an implementation.

FIG. 6 is a manual creator of registration points on the medical hologram 600, according to an implementation. The creator 600 is one implementation of the creator 150 in FIG. 1. The creator 600 includes a recognizer of finger position 610 that is operably coupled to a tracker 620 of the finger position. In some implementations, the finger tracking software development toolkit of MRTK (Mixed Reality Toolkit) from Windows is used to recognize the user's finger.

Once the finger is tracked, a medical hologram of a point is projected on top of the finger as follows:

The user can then determine the registration points on the medical hologram themselves one by one. To place a point on the medical hologram, the user needs to bring their finger to the desired position of a marking point and then gives the speech command “Set” or “Place”.

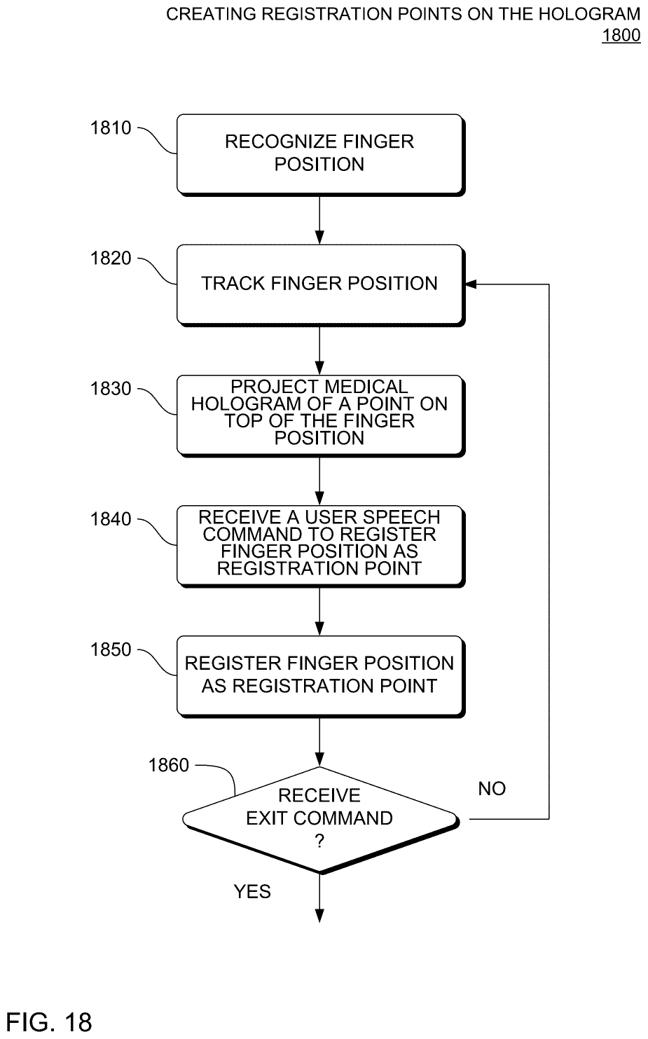

The tracker 620 is operably coupled to a projector 630 on a medical hologram of a point on top of the finger position. The projector 630 is operably coupled to a receiver 640 of a command to register finger position as a registration point. The receiver 640 is operably coupled to a registrar 650 of finger position as a registration point. The registrar 650 is operably coupled to an evaluator 660 of a command. If the evaluator 660 determines that the command is not an exit command, the tracker 620 is activated to continue processing the finger position by the tracker at 620 and registering the finger position as a registration point by registrar 650. In some implementations, the creator 600 performs method 1800 in FIG. 18.

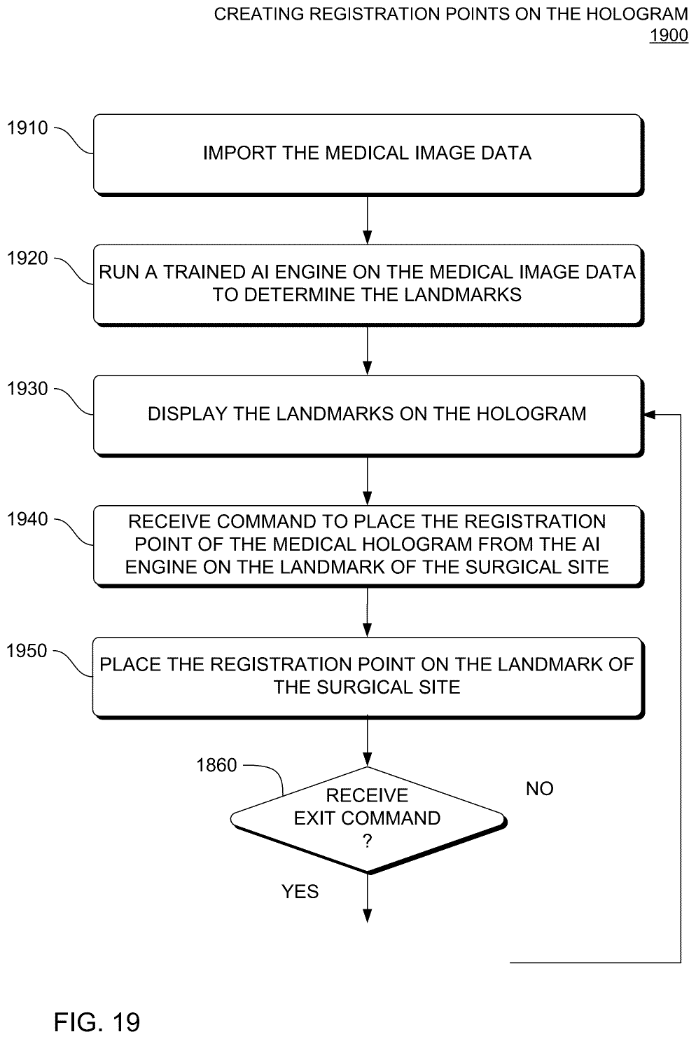

FIG. 7 is an automatic creator of registration points on the medical hologram 700, according to an implementation. The automatic creator 700 is one implementation of the creator 150 in FIG. 1. The automatic creator 700 includes an importer of medical image data, at block 710, that is operably coupled to a trained artificial intelligence engine, at block 720, that is operably coupled to a displayer 730 of the landmarks on the hologram, that is operably coupled to a receiver 740 of a command to register a position as a registration point, that is operably coupled to a registrar 750 of the position as a registration point on the surgical site. The registrar 750 is operably coupled to an evaluator 660 of a command. If the evaluator 660 determines that the command is not an exit command, the displayer 730 is activated to continue displaying landmarks on the hologram, receiving a new command 740 and registering the finger position as a registration point by registrar 750. In some implementations, the automatic creator 700 performs method 1900 in FIG. 19.

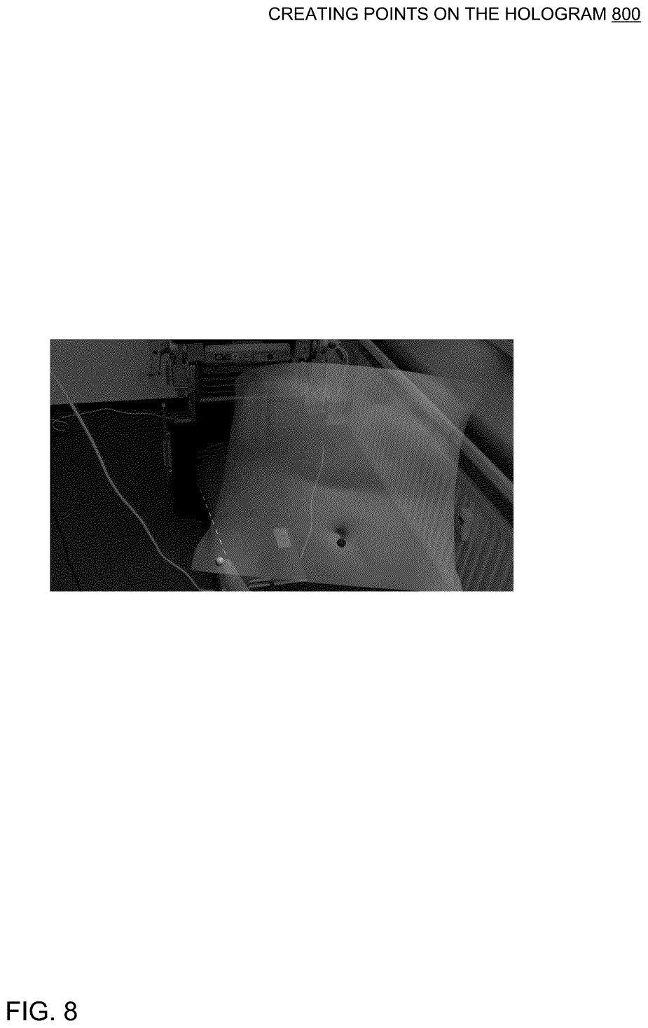

FIG. 8 is a graphic user interface of creating points on the medical hologram 800, according to an implementation.

FIG. 9 is a graphical user interface showing the next button for point placement on a surgical site 900, according to an implementation.

FIG. 10 is a block diagram of a placer 1000 of registration points on a surgical site, according to an implementation. The placer 1000 is one implementation of the placer 170. The placer 1000 includes a displayer of a next created registration point on the medical hologram 1010. The displayer 1010 is operably coupled to a finger position tracker 1020. The tracker 1020 is operably coupled to a projector of the registered point of the medical hologram on the finger position 1030. The projector 1030 is operably coupled to a receiver of a command to place the registered point of the medical hologram on the finger on the landmark of the surgical site 1040. The receiver 1040 is operably coupled to a placer of the next registration point of the medical hologram on the finger as a registration point on the landmark of the surgical site. The placer 1050 is operably coupled to an evaluator of a registered point on the medical hologram 1060. If the evaluator 1060 indicates that there is a registered point on the medical hologram, the tracker 1020 is activated to continue processing the registered point on the medical hologram and placing the registered point of the medical hologram on the finger as a registration point on the landmark of the surgical site 1050.

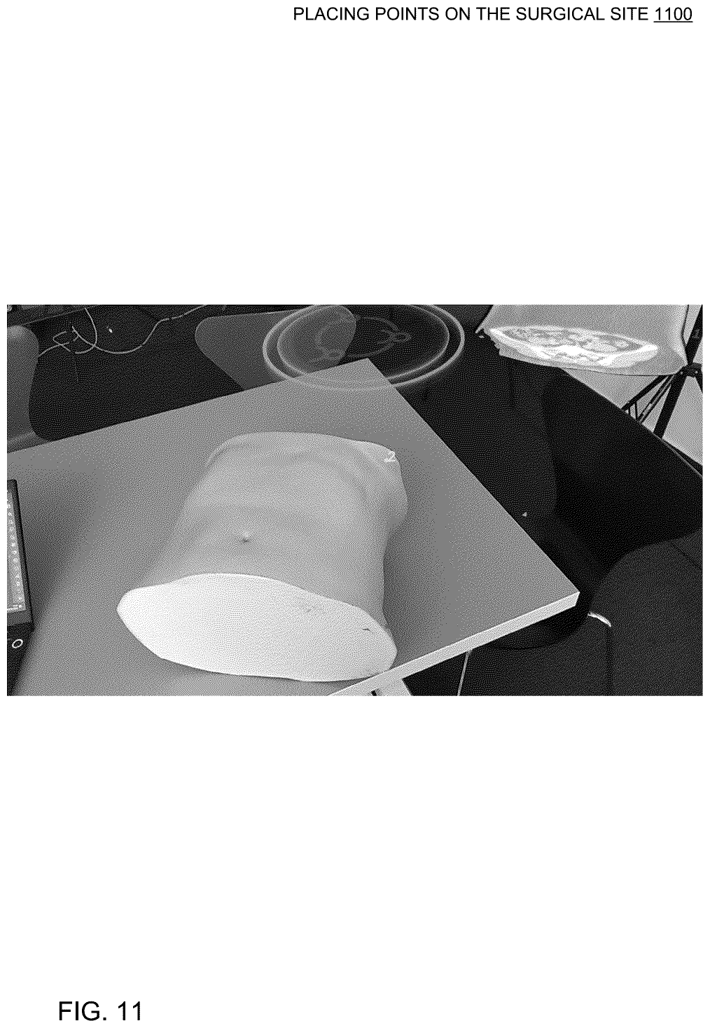

FIG. 11 is a graphical user interface of placing points on the surgical site 1100 according to an implementation that is performed by FIG. 10.

FIG. 12 is a block diagram of a registrar 1200 of the medical hologram on a surgical site, according to an implementation. The registrar 1200 is one implementation of the registrar 190.

Apparatus 1200 includes a determiner 1210 of centroids of both point sets. Determining the centroids of both point sets A and B is performed as follows:

$${centroid}_{A} = \frac{1}{N} {\sum }_{i = 1}^{N} {A}^{i}$$

$${centroid}_{B} = \frac{1}{N} {\sum }_{i = 1}^{N} {B}^{i}$$

Apparatus 1200 also includes a re-centerer 1220 of both set points so that both set points share the same origin, as shown below:

Apparatus 1200 includes calculator 1230 of the covariance matrix H, as shown below:

$$H = ( A - {centroid}_{A} ) {( B - {centroid}_{B} )}^{T}$$

Apparatus 1200 also includes a singular value decomposition (SVD) library 1240 to find the rotation matrix R=VU^{T}, as shown below:

$$[ U , S , V ] = SVD ( H )$$

SVD is conventionally described as the factorization of a 2D matrix. The purpose of the SVD implementation is to find the optimal rotation and translation between two set of points: Reference points from the medical hologram and target points on the surgical site. The transformation matrix T (4×4) consists of a (3×3) rotation matrix and a translation vector T.

This is a rigid transformation because it preserves the shape and size. We use the term “optimal” because the data is noisy and the least square error will be minimized, as shown below:

$$err = {\sum }_{i = 1}^{N} {‖ {RA}^{i} + t - {B}^{i} ‖}^{2}$$

One example of a SVD function that can be used is numpy.linalg.svd( )published in the Numpy open source library by the Numpy Developers and described at https://numpy.org/doc/stable/reference/generated/numpy.linalg.svd.html.

Apparatus 1200 includes a determiner 1250 of an optimal rotation. Apparatus 1200 also includes an inserter 1260 of the found parameters to the general translation equation to find values which minimize the least square error. Apparatus 1200 includes a determiner 1270 of the translation vector T, and apparatus 1200 includes a transmitter 1280 of the rotation matrix R and the translation vector T.

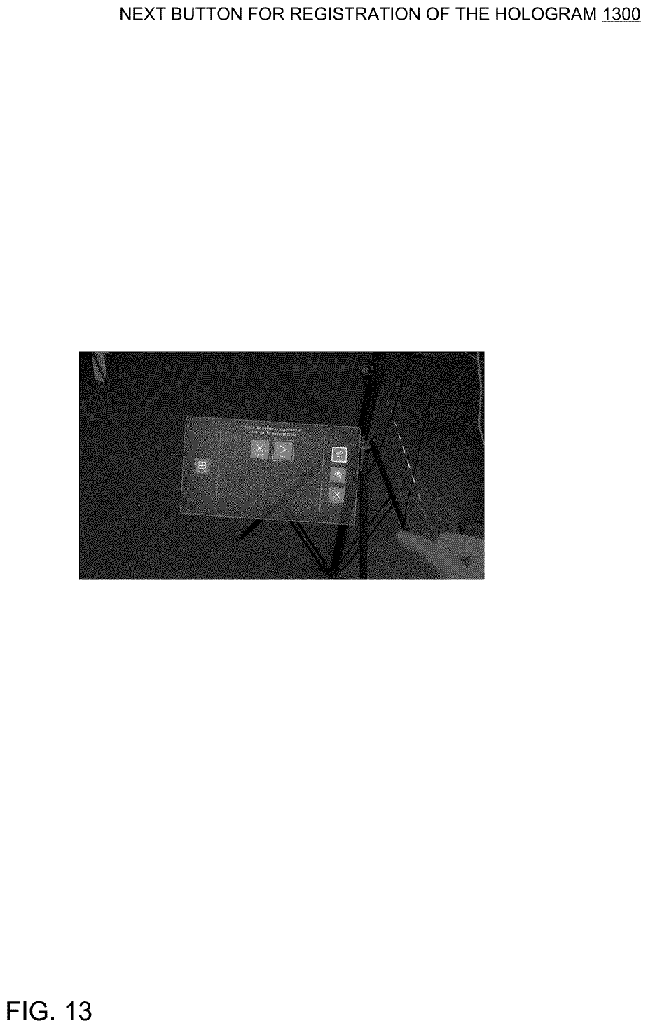

FIG. 13 is a graphical user interface of a next button for registration of the medical hologram 1300, according to an implementation that is performed by FIG. 12.

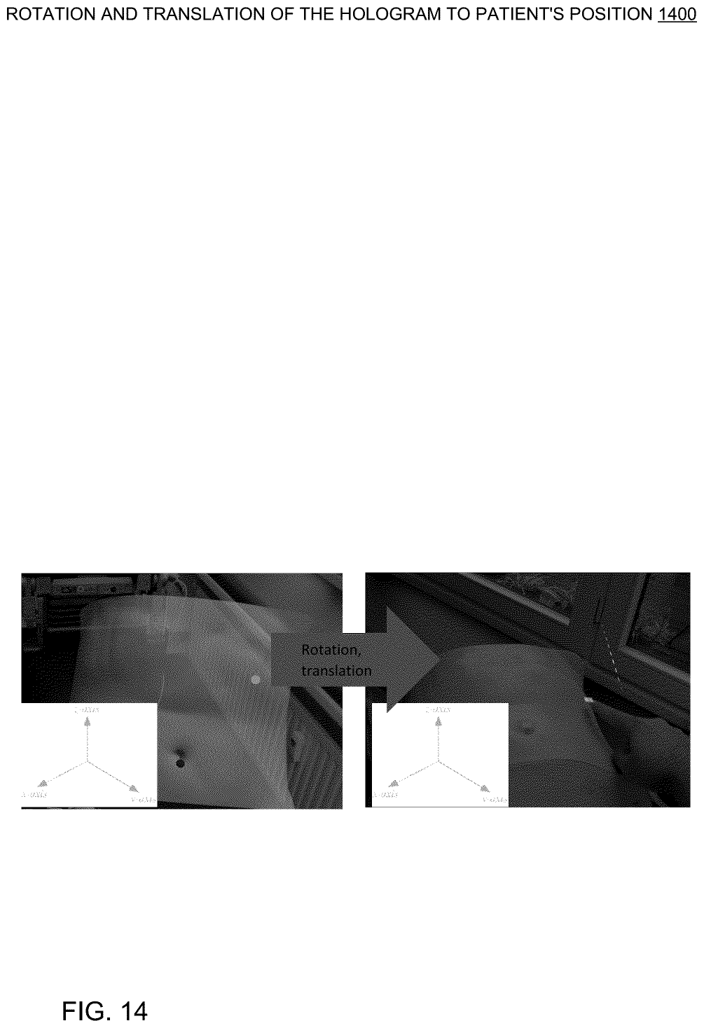

FIG. 14 is a graphical user interface of rotation and translation of the medical hologram to the patient's position 1400, according to an implementation that is performed by FIG. 12.

### Method Implementations

In the previous section, apparatus of the operation of an implementation was described. In this section, the particular methods performed by system 100 in FIG. 1 and apparatus 200 in FIG. 2, apparatus 400 in FIG. 4, apparatus 600 in FIG. 6, apparatus 1000 in FIG. 10 and apparatus 1200 in FIG. 12, of such an implementation are described by reference to a series of flowcharts.

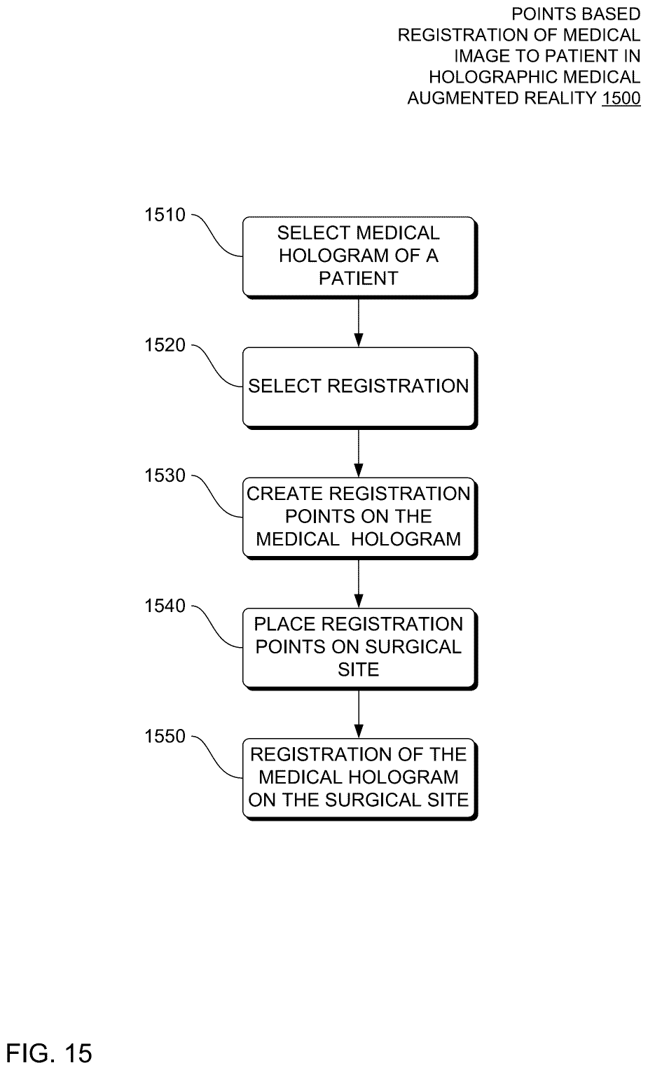

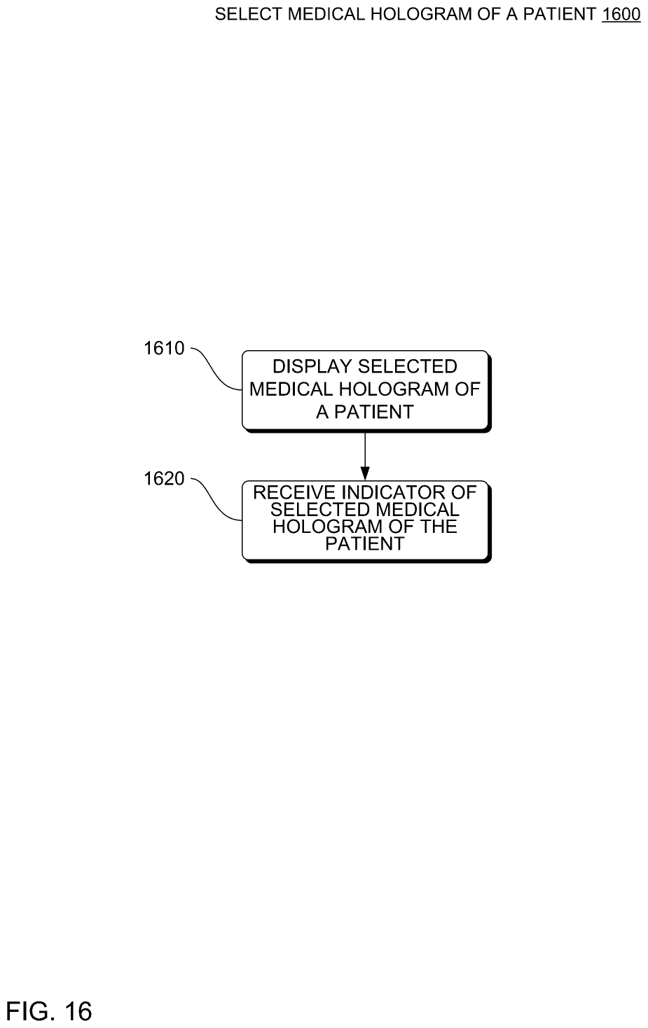

FIG. 15 is a flowchart of a method of points-based registration of a medical hologram to a surgical site in holographic medical augmented reality 1500, according to an implementation. Method 1500 includes selecting a medical hologram of a patient, at block 1510. One example of selecting 1510 is shown in FIG. 16.

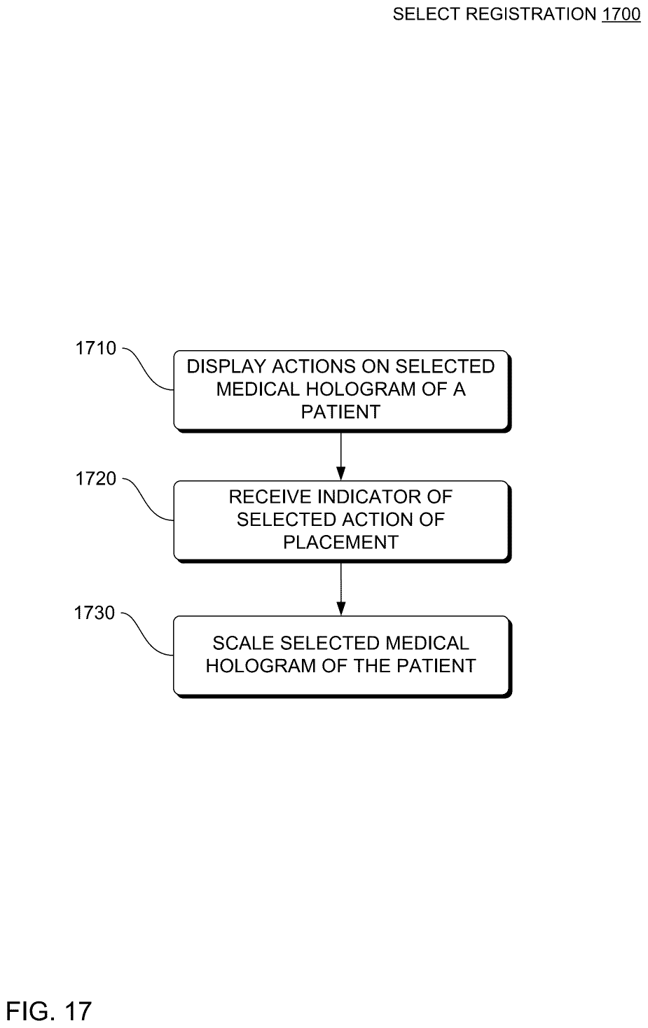

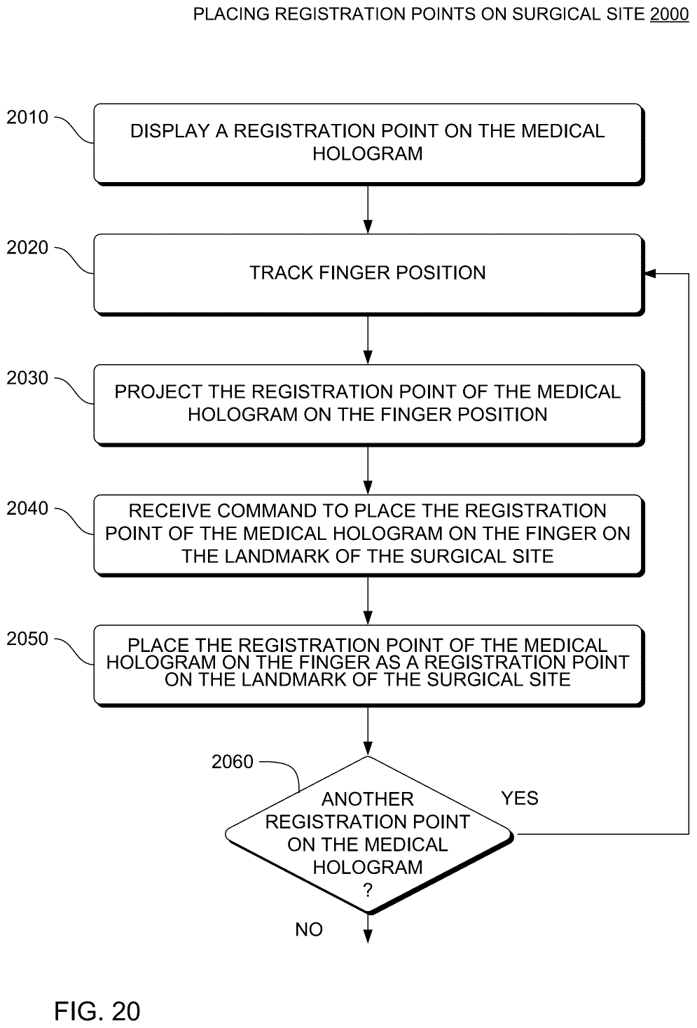

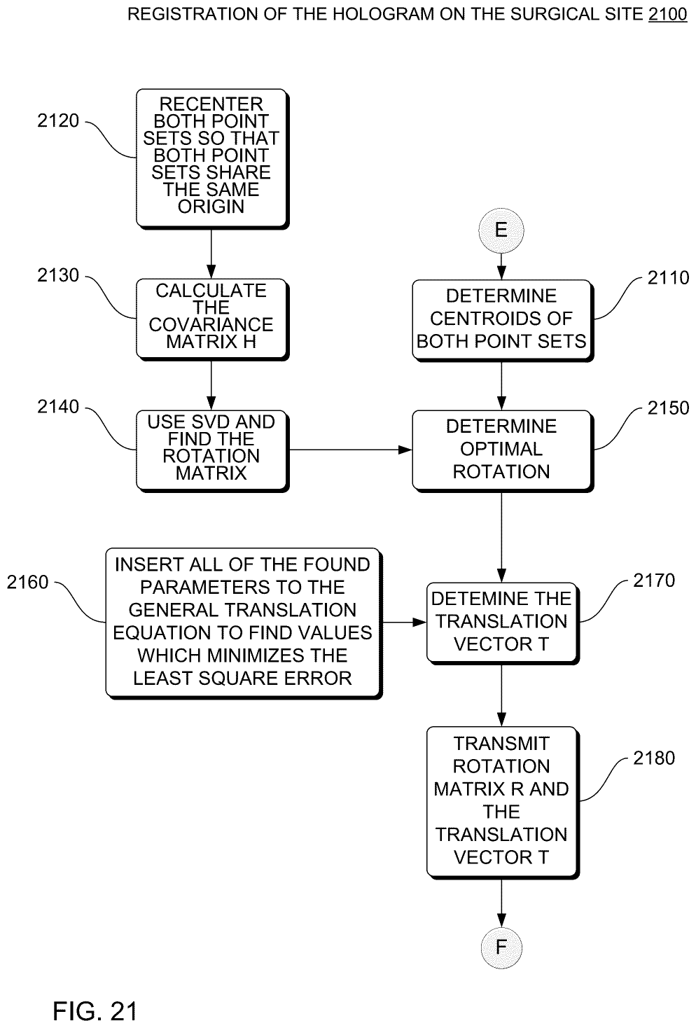

Once a medical hologram of a patient is selected, the user can start the point-based registration process by selecting “placement” from the object toolbox, accordingly, in some implementations, method 1500 includes selecting registration at block 1520. One example of selecting registration 1520 is FIG. 17. Thereafter method 1500 includes creating registration points on the medical hologram at block 1530. One example of creating registration points on the medical hologram 1530 is FIG. 18. Another example of creating registration points on the medical hologram 1530 is FIG. 19. Thereafter method 1500 includes placing registration points on a surgical site, at block 1540. One example of placing registration points on the surgical site 1540 is in FIG. 20. Thereafter method 1500 includes registering the medical hologram on the surgical site, at block 1550. One example of superimposition of the medical hologram on the surgical site 1550 is FIG. 21. An apparatus to perform method 1500 is shown in FIG. 1.

FIG. 16 is a flowchart of a method 1600 to select a medical hologram of a patient, according to an implementation. Method 16 is one implementation of selecting a medical hologram of a patient 1510 in FIG. 15. Method 16 includes displaying a medical hologram of a patient, and at block 1610 and then receiving an indicator of the selected medical hologram of a patient, at block 1620. Method 1600 can be performed by apparatus 200 in FIG. 2. Method 1600 generates the indicator 120 of the selected medical hologram of a patient in FIG. 1.

FIG. 17 is a flow chart of a method of selecting registration 1700, according to an implementation. Method 1700 is one implementation of selecting registration 1520 in FIG. 15. Method 1700 includes displaying actions on a selected medical hologram of a patient, at block 1710, and then receiving an indicator of a selected action of placement, at block 1720 and then scaling the selected medical hologram of a patient, at block 1730. In some implementations, the anatomical part of a patient is scaled to 1:1 size, to make sure that registration happens with the correct geometric dimensions. Method 1700 can be performed by the registration selector 130 and FIG. 1 or the apparatus 400 in FIG. 4. Method 1500 generates the indicator 140 of replacement in FIG. 1

FIG. 18 is a flow chart of a method of manually creating registration points on the medical hologram 1800, according to an implementation. Method 1800 is one implementation of creating registration points on the medical hologram 1530 in FIG. 15. Method 1800 includes recognizing a finger position at block 1810, and then tracking the finger position at block 1820. In some implementations, the finger tracking software development toolkit of MRTK (Mixed Reality Toolkit) from Windows is used to recognize the user's finger.

Once the finger is tracked, a medical hologram of a point is projected on top of the finger as follows:

Method 1800 includes projecting a medical hologram of a point on top of the finger position, at block 1830, and then receiving a user speech command to register the finger position as a registration point, at block 1840, and then registering the finger position as a registration point, at block 1850. Thereafter method 1800 includes determining whether an exit command has been received, at block 1860 and if no exit command has been received the method 1800 continues at tracking a finger position at block 1820, otherwise when an exit command is received, the method 1800 ends. In some implementations method 1800 can be performed by the creator of registration points on the medical hologram at block 150 in FIG. 1 or by the creator of registration points on the medical hologram 600 in FIG. 6. Method 1800 generates the indicators of created registration points 160 in FIG. 1.

FIG. 19 is a flow chart of a method of automatically creating registration points on the medical hologram 1900, according to an implementation. Method 1900 is one implementation of creating registration points on the medical hologram 1530 in FIG. 15. Method 1900 includes importing medical image data, at block 1910, and then operating a trained artificial intelligence (AI) engine on the medical image data to determine the landmarks, at block 1920. For example, in the case of a head scan, the AI engine can determine a set of suggested landmarks on a scalp. Thereafter, method 1900 include displaying the landmarks on the hologram, at block 1930 and then receiving a command to place the registration point of the medical hologram from the AI engine on the landmark of the surgical site, at block 1940, and then placing the registration point indicated in the receiving command of 1940, on the landmark of the surgical site, at block 1950. Thereafter method 1900 includes determining whether an exit command has been received, at block 1860 and if no exit command has been received the method 1900 continues at displaying 1930 the landmarks on the hologram, otherwise when an exit command is received, the method 1900 ends. In some implementations method 1900 can be performed by the creator of registration points on the medical hologram at block 150 in FIG. 1 or by the creator of registration points on the medical hologram 700 in FIG. 7. Method 1900 generates the indicators of created registration points 160 in FIG. 1.

FIG. 20 is a flow chart of a method of placing registration points on a surgical site 2000, according to an implementation. Method 2000 is one example of placing registration points on a surgical site 1540 in FIG. 15. Method 2000 includes displaying a registered point on the medical hologram, at block 2010, and then tracking a finger position, and at block 2020. Thereafter method 2000 includes projecting the registered point of the medical hologram on the finger position, at block 2030, and then receiving a command to place the registered point of the medical hologram on the finger on the landmark of the surgical site, at block 2040, and then placing the registered point of the medical hologram on the finger as a registration point on the landmark of the surgical site, and at block 2050. Thereafter method 2000 includes determining whether another created registration point exists on the medical hologram at block 2060, and if another created registration point on the medical hologram exists then the method continues at tracking the finger position at block 2020, otherwise if there is not another created registration point on the medical hologram the method 2000 ends. Method 2000 can be performed by the placer 170 of registration points on the surgical site in FIG. 1 or by the placer 1000 of registration points on the surgical site in FIG. 10. Method 2000 generates the indicator 180 of placed registration points on the surgical site in FIG. 1.

FIG. 21 is a method of registration of the medical hologram on the surgical site 2100, according to an implementation. Method 2100 is one implementation of registration of the medical hologram on the surgical site at block 1550 and FIG. 15. Method 2100 can be performed by the registrar 190 of the medical hologram on the surgical site in FIG. 1 or by the registrar of the medical hologram on a surgical site 1200 in FIG. 12. Method 2100 generates the indicator of the placed registration points on the surgical site 195 in FIG. 1.

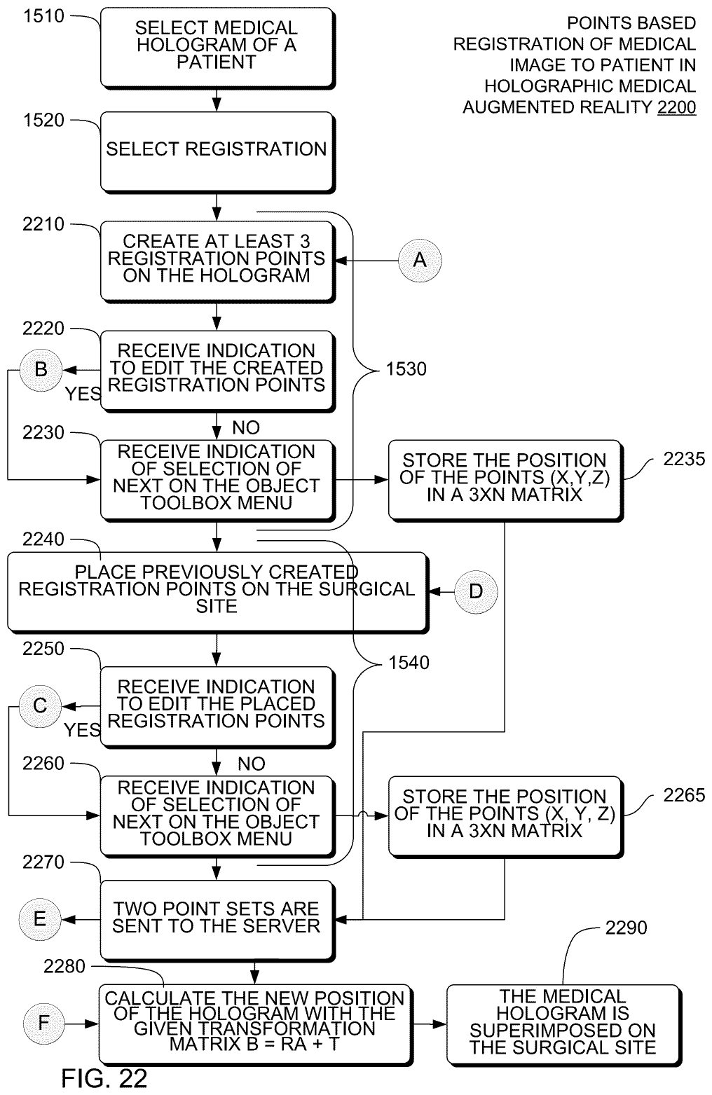

FIG. 21 is a flowchart of a method 2100 of registering the medical hologram on the surgical site, according to an implementation. Method 2100 is entered at entry point E from FIG. 22, and method 2100 includes determining centroids of both point sets, at block 2110. Determining the centroids of both point sets A and B is performed as follows

$${centroid}_{A} = \frac{1}{N} {\sum }_{i = 1}^{N} {A}^{i}$$

$${centroid}_{B} = \frac{1}{N} {\sum }_{i = 1}^{N} {B}^{i}$$

Method 2100 also includes re-centering both set points so that both set points share the same origin, at block 2120, as shown below:

Thereafter method 2100 includes calculating the covariance matrix H, at block 2130, as shown below:

$$H = ( A - {centroid}_{A} ) {( B - {centroid}_{B} )}^{T}$$

Thereafter method 2100 includes using singular value decomposition (SVD) to find the rotation matrix R=VU^{T}, at block 2140. as shown below:

$$[ U , S , V ] = SVD ( H )$$

SVD is conventionally described as the factorization of a 2D matrix. The purpose of the SVD implementation is to find the optimal rotation and translation between two set of points: Reference points from the medical hologram and target points on the surgical site. The transformation matrix T (4×4) consists of a (3×3) rotation matrix and a translation vector T.

### Dataset A Dataset B

This is a rigid transformation because it preserves the shape and size We use the term “optimal” because the data is noisy and the least square error will be minimized, as shown below:

$$err = {\sum }_{i = 1}^{N} {‖ {RA}^{i} + t - {B}^{i} ‖}^{2}$$

One example of a SVD function that can be used is numpy.linalg.svd( ) published in the Numpy open source library by the Numpy Developers and described at
https://numpy.org/doc/stable/reference/generated/numpy.linalg.svd.html.

After blocks 2110 and 2140 have been performed method 2100 includes determining an optimal rotation, at block 2150. Method 2100 also includes inserting all the found parameters to the general translation equation to find values which minimize the least square error, at block 2160. After blocks 2150 and 2160 have been performed, method 2100 includes determining the translation vector T, and thereafter method 2100 includes transmitting the rotation matrix R and the translation vector T, at block 2180.

FIG. 22 is a flowchart of a method 2200 of points-based registration of a medical hologram to a surgical site in holographic medical augmented reality 2200, according to an implementation. Method 2200 includes selecting a medical hologram of a patient, at block 1510. One example of selecting 1510 is shown in FIG. 16. Once a medical hologram of a patient is selected, the user can start the point-based registration process by selecting “placement” from the object toolbox, accordingly, in some implementations, method 2200 includes selecting registration at block 1520. One example of selecting registration 1520 is FIG. 17. Thereafter method 2200 includes creating at least 3 registration points on the medical hologram at block 2210. One example of creating registration points on the medical hologram 2210 is FIG. 18.

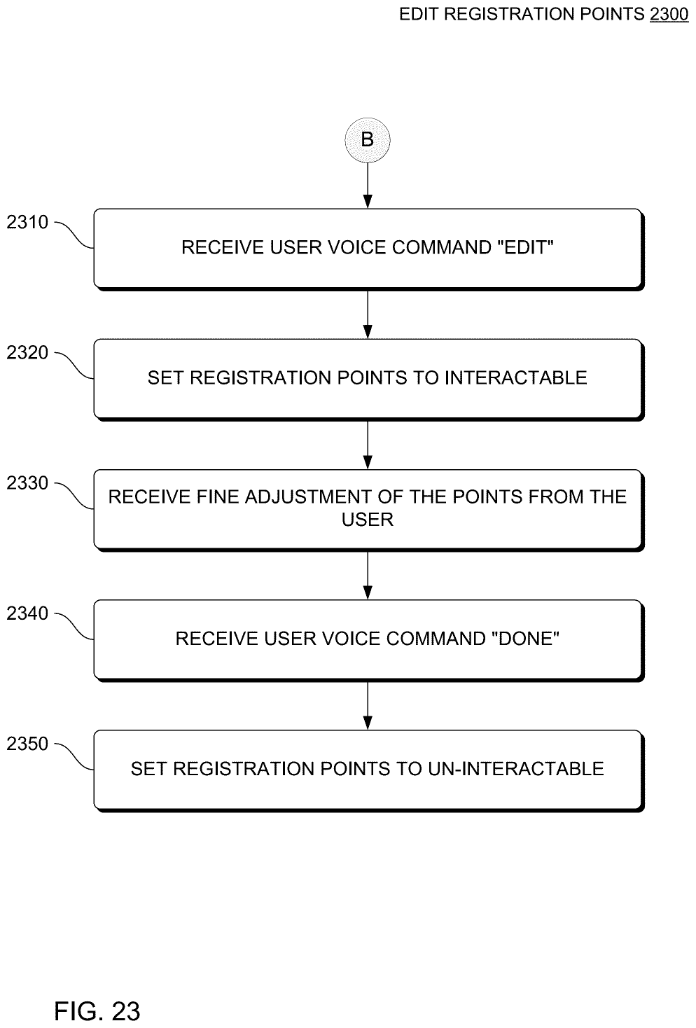

Thereafter method 2200 includes determining whether an indication from a user to edit the created registration points has been received, at block 2220. If the indication of the user indicating to edit the created registration points is “yes” then the method continues at Point B in FIG. 23. If the indication of the user to edit the registration point is “no” then the method 2200 continues by receiving an indication of a selection of next on the object toolbox menu, at block 2230. Thereafter method 2200 continues by storing the position of the points XYZ in a 3XN matrix, at block 2235 in which in Reference Point Set (Matrix A), p1 p2 p3 . . . pn−−>min 3 points are required; x1 x2 x3 . . . xn; y1 y2 y3 . . . yn; and z1 22 z3 . . . zn. Theoretically there is no limit for maximum number of points, however for the practical reasons surgeons tend to create maximum 6 to 7 points. After block 2235 is performed, the method 2200 continues at block 2270. Blocks 2210, 2220 and 2230 are similar to step 1530 in FIG. 15.

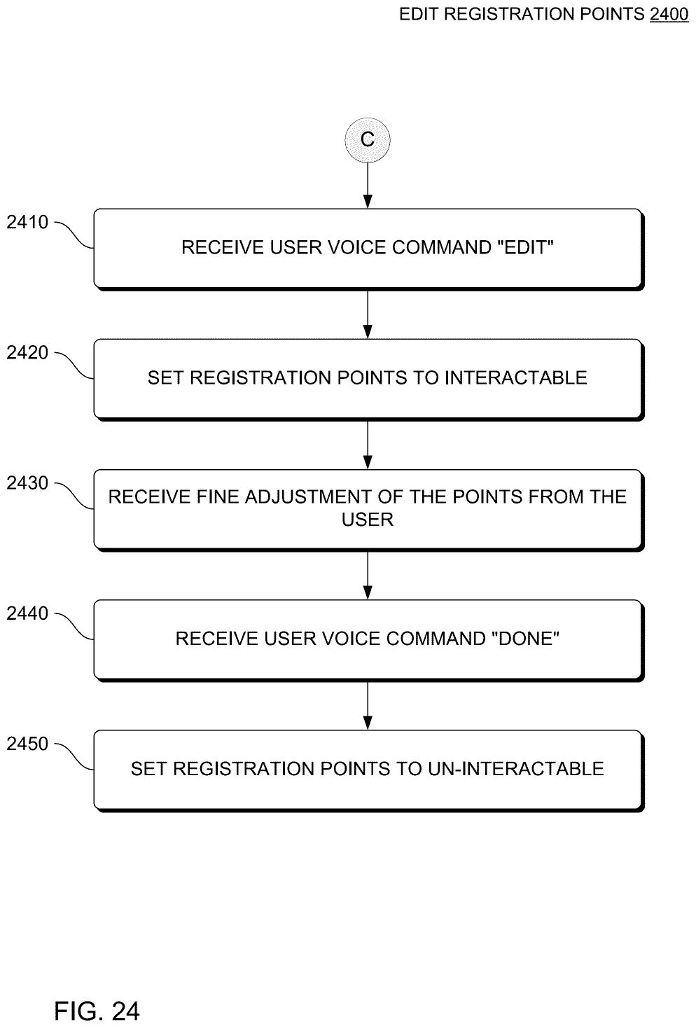

After block 2230 is performed, the method 2200 continues by placing previously created registration points on the surgical site, at block 2240. After performance of block 2240, the method 2200 continues by determining whether an indication from a user to edit the created registration points has been received, at block 2250. If the indication of the user indicating to edit the created registration points is “yes” at block 2250 then the method 2200 continues at Point C in FIG. 24. If the indication of the user to edit the creation points is “no” at block 2250 then the method 2200 continues. If the indication of the user to edit the creation points is “no” then the method 2200 continues by receiving an indication of a selection of next on the object toolbox menu, at block 2260. Thereafter method 2200 continues by storing the position of the points XYZ in a 3XN matrix, at block 2265 in which in Reference Point Set (Matrix B), p1 p2 p3 . . . pn−−>the same number of points at block 2235; x1 x2 x3 . . . xn; y1 y2 y3 . . . yn; and z1 z2 z3 . . . zn. After block 2265 is performed, the method 2200 continues at block 2270. Blocks 2240, 2250 and 2260 are similar to step 1540 in FIG. 15.

After block 2260 is performed, the method 2200 continues by determining whether 2 point sets are sent to the server, at block 2270. If the 2 point sets are sent to the server, the method 2200 continues by performing method 2100 in FIG. 21. If the 2 point sets are not sent to the server, method 2200 continues by calculating the new position of the medical hologram with the given transformation matrix b equals RA+T, at block 2280 and thereafter method 2200 continues by superimposing the medical hologram on the surgical site, at block 2290. Blocks 2270, 2280 and 2290 are similar to block 1550 in FIG. 15.

FIG. 23 is a flow chart of a method 2300 of editing registration points, according to an implementation. Method 2300 is invoked at Point B in FIG. 22. Method 2300 includes receiving a voice command from a user to edit, at block 2310. Thereafter method 2300 includes setting registration points to interactable, at block 2320. Thereafter method 2300 includes receiving fine adjustments of the points from the user, at block 2330. Thereafter method 2300 includes receiving a voice command from the user “done”, at block 2140. Thereafter method 2300 includes setting the registration points to un-interactable, at block 2350.

FIG. 24 is a flow chart of a method 2400 of editing registration points, according to an implementation. Method 2400 is invoked at Point C in FIG. 22. Method 2400 includes receiving a voice command from a user to edit, at block 2410. Thereafter method 2400 includes setting registration points to interactable, at block 2420. Thereafter method 2400 includes receiving fine adjustments of the points from the user, at block 2430. Thereafter method 2400 includes receiving a voice command from the user “done”, at block 2240. Thereafter method 2400 includes setting the registration points to un-interactable, at block 2450.

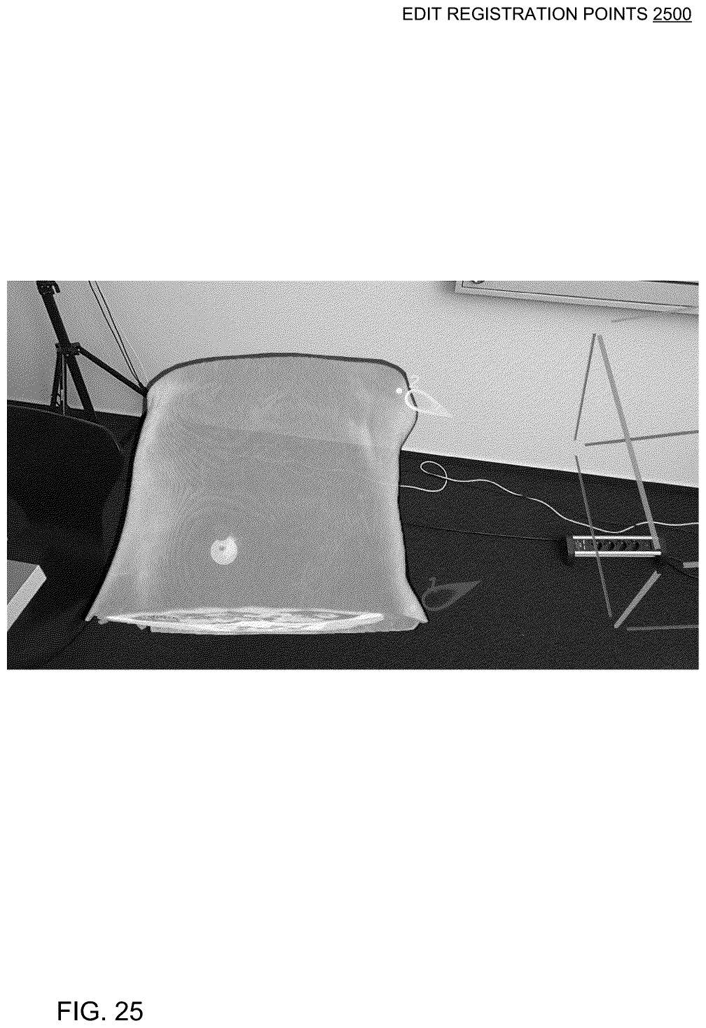

FIG. 25 is a graphical user interface 2500 showing the editing of registration points on a surgical site, according to an implementation

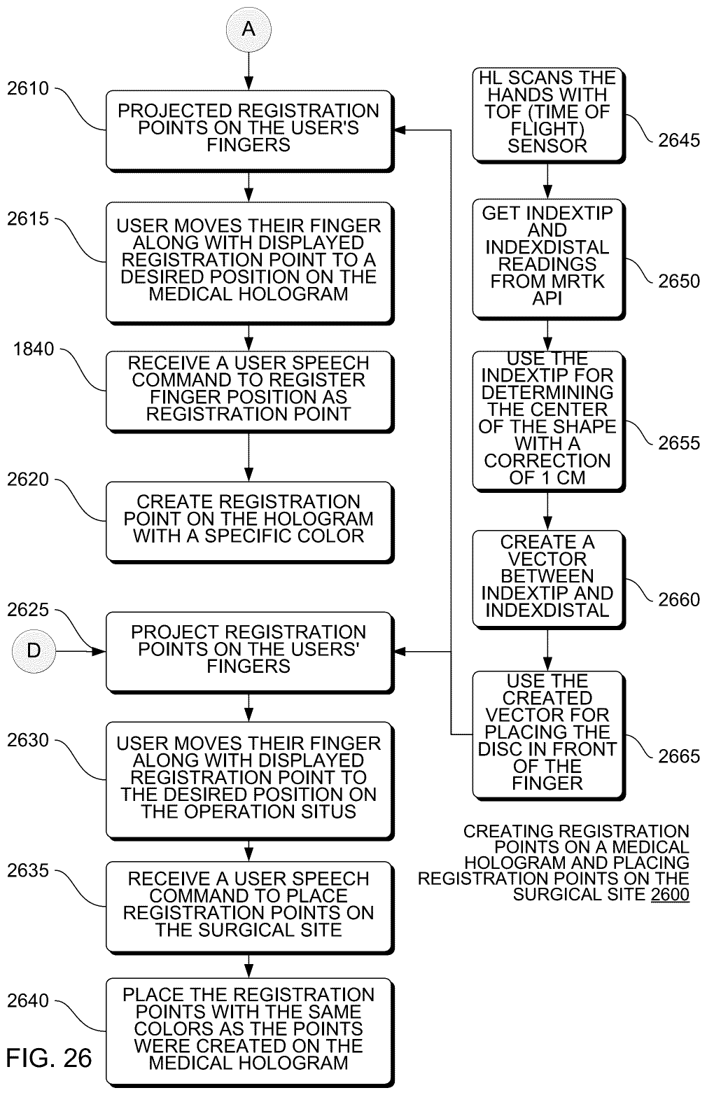

FIG. 26 is flowcharts of two methods of creating registration points on a medical hologram and placing registration points on the surgical site 2600, according to an implementation. The first method of 2600 of creating registration points on a medical hologram can be entered from entry point A from FIG. 22. The first method of 2600 includes projecting registration points on the user's fingers, at block 2610 and thereafter the user moves their finger along with displayed registration point to the desired position on the medical hologram, at block 2615 and thereafter method 2600 includes receiving a user speech command to register finger position as a registration point, at block 1840 and thereafter method 2600 includes creating a registration point on the hologram with a specific color, at block 2620. The first method of 2600 includes blocks 2610, 2615, 1840 and 2620. The second method of 2600 starts either from entry point D in FIG. 22 or from block 2665 in FIG. 26, in which registration points are projected on the user's fingers at block 2625. Thereafter method 2600 includes receiving a user speech command to place registration points on the surgical site, at block 2635 and thereafter method 2600 includes placing the registration points with the same colors as they were created on the medical hologram, at black 2640. The second method of 2600 of placing registration points on the surgical site includes blocks 2625, 2630, 2635 and 2640.

Method 2600 also includes performing HL scans with the hands time of flight (TOF) sensor, at block 2645, retrieving an index tip and interdistal readings from an MRTK application interface, at block 2650, using the index tip for determining the center of the shape with the correction of about 1 cm, at block 2655, creating a vector between the index tip and the index distal, at block 2660, and thereafter using the created vector for placing the disc in front of the finger, at block 2665.

### UML

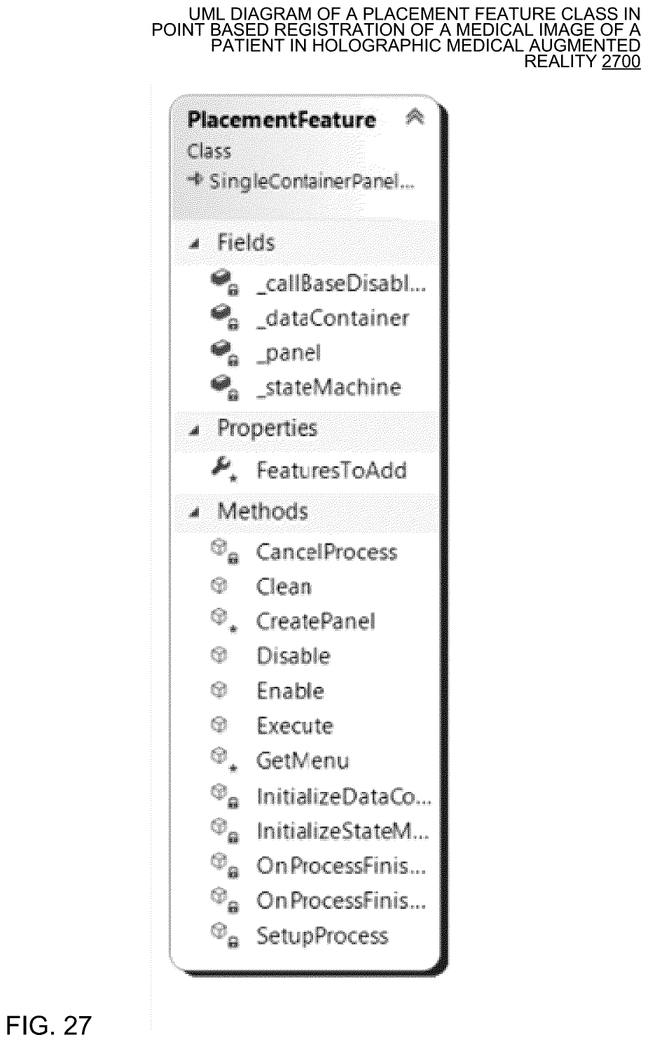

FIG. 27 is a diagram of a placement feature class 2700, according to an implementation. The placement feature class 2700 is instantiated from single container panel class.

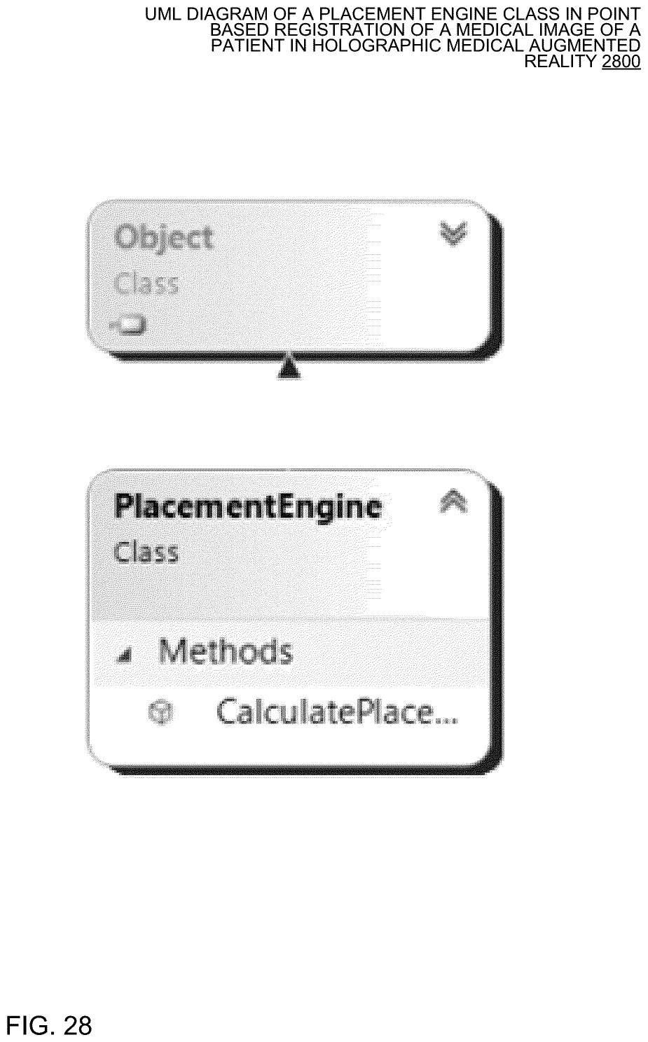

FIG. 28 is a diagram of a placement engine class 2800 according to an implementation. The placement engine class is a subclass of the object class.

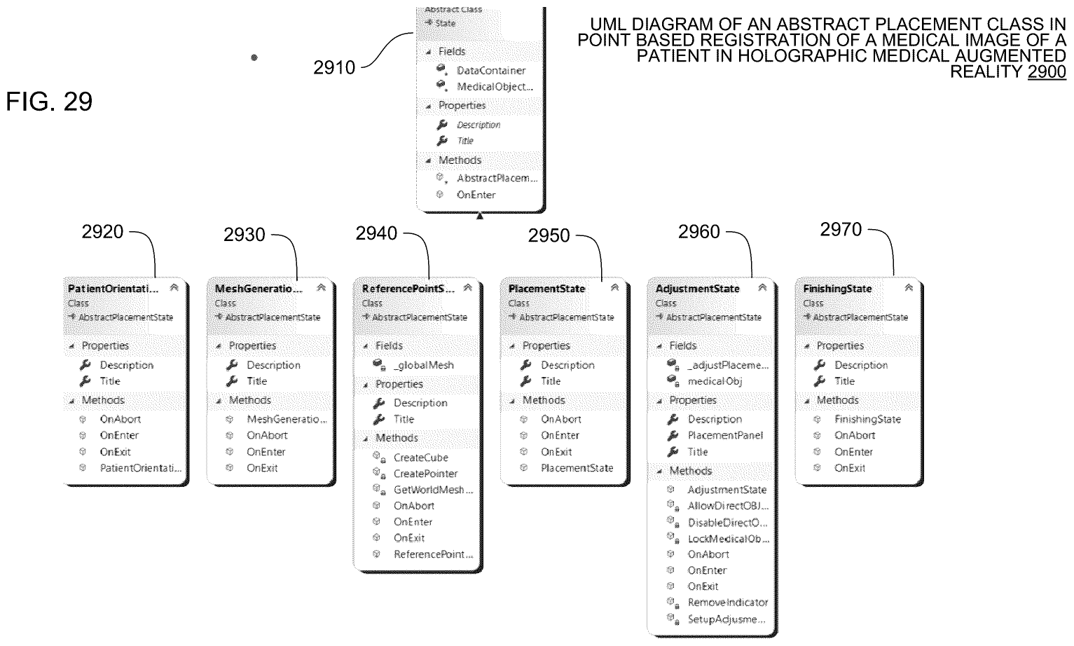

FIG. 29 is a diagram of the abstract placement class, according to an implementation. The abstract placement class 2610 has six different child classes, including a patient orientation class 2920 a mesh generation class 2930, a reference point class 2940, a placement state 2950, and adjustment state class 2960 and a finishing state class 2970.

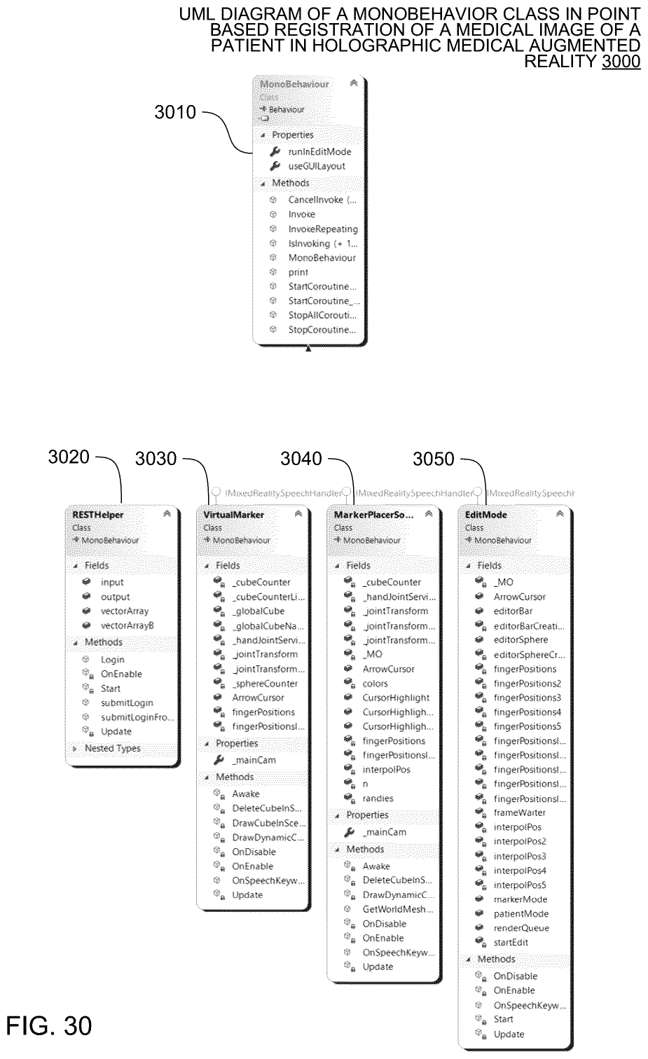

FIG. 30 is a diagram of a monobehavior class 3000, according to an implementation. The monobehavior class 3010 includes at least four subclasses, such as a RESThelper class 3020, a virtual marker class 3030, a marker placer sophisticated class 3040 and an edit mode class 3050.

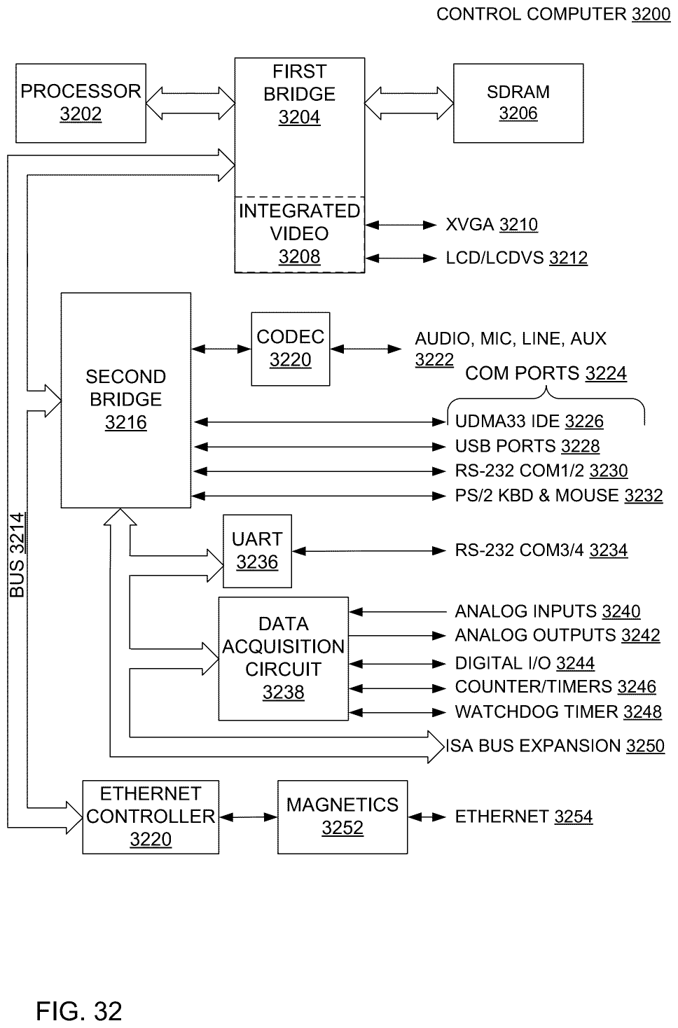

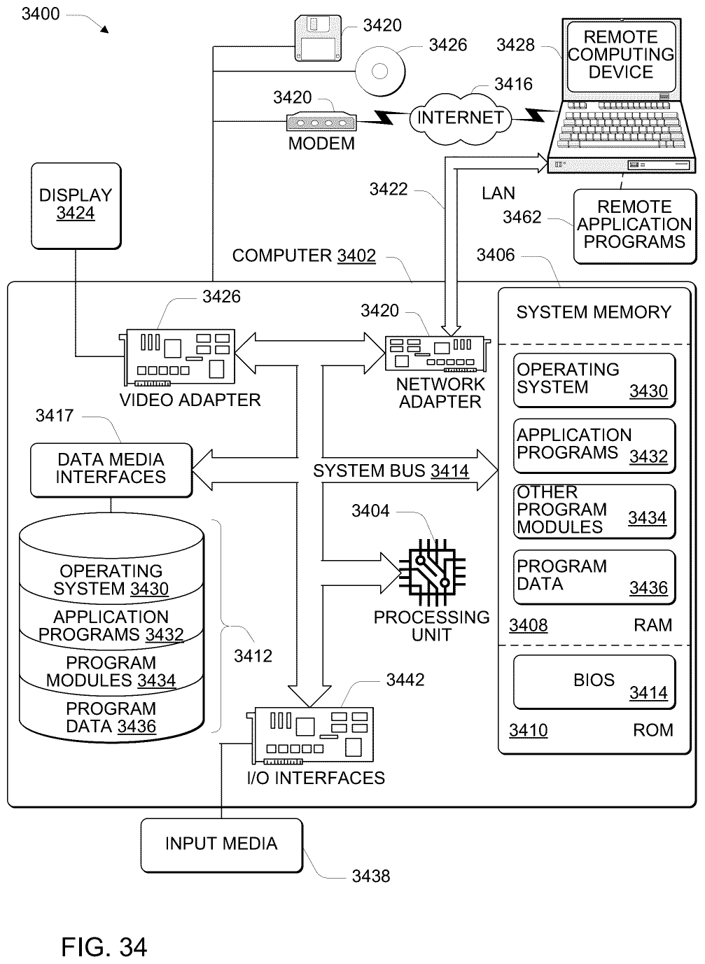

In some implementations, methods 1500-2600 and UML classes 2700-3000 are implemented as a sequence of computer instructions which, when executed by a processor, such as processor 3202 in FIG. 32, processing unit 3404 in FIG. 34 or main processor 3502, cause the processor to perform the respective method. In other implementations, methods 1500-2600 and UML classes 2700-3000 are implemented as a computer-accessible medium having executable instructions capable of directing a processor, such as processing unit 3404 in FIG. 34, to perform the respective method. In varying implementations, the medium is a magnetic medium, an electronic medium, or an optical medium.

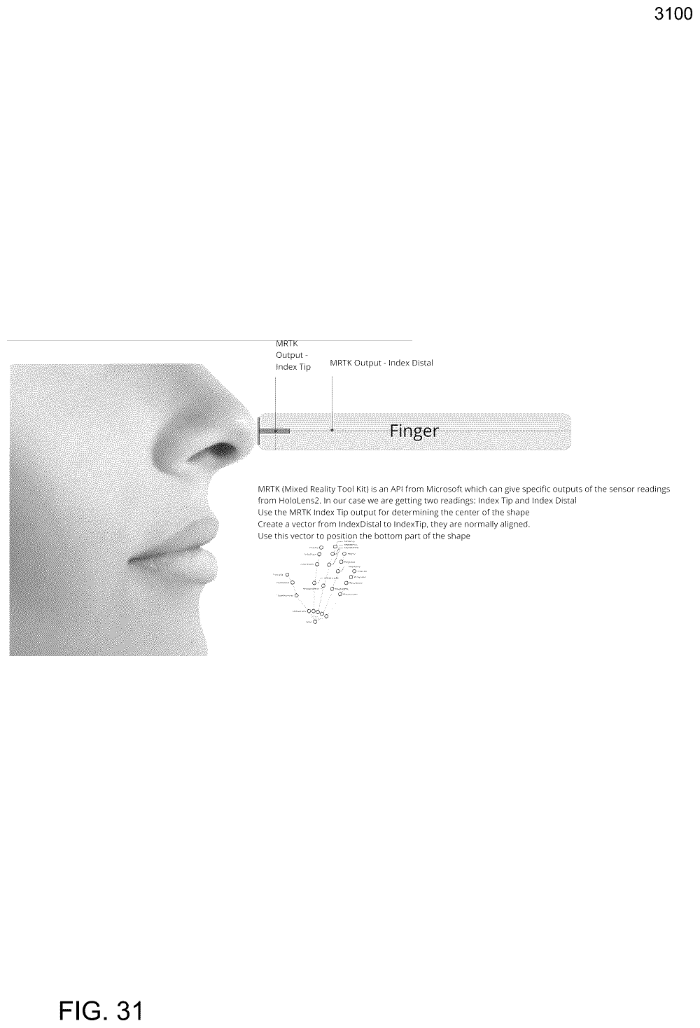

FIG. 31 is a diagram 3100 describing finger tracking, according to an implementation.

### Hardware and Operating Environments

FIG. 32 is a block diagram of a holographic medical augmented reality control computer 3200 in which different implementations can be practiced. The holographic medical augmented reality control computer 3200 includes a processor 3202 (such as a Pentium III processor from Intel Corp. in this example) which includes dynamic and static ram and non-volatile program read-only-memory (not shown), a first bridge 3204, operating memory 3206 (SDRAM in this example). The first bridge 3204 includes integrated video 3208 that couples the holographic medical augmented reality control computer 3200 to a XVGA communication path 3210 and an LCD and/or LCDVS device 3212.

The first bridge 3204 is operably coupled to a bus 3214 and the bus 3214 is operably coupled to a second bridge 3216 and an Ethernet® controller 3218.

The second bridge 3216 is operably coupled to a CODEC 3220 and the CODEC 3220 is coupled to an audio port 3222. The second bridge 3216 is operably coupled to communication ports 3224 (e.g., UDMA IDE 3226, USB port(s) 3228, RS-232 3230 COM1/2 and/or keyboard interface 3232).

An RS-232 port 3234 is coupled through a universal asynchronous receiver/transmitter (UART) 3236 to the second bridge 3216.

The second bridge 3216 is operably coupled to a data acquisition circuit 3238 with analog inputs 3240 and outputs 3242 and digital inputs and outputs 3244.

In some implementations of the holographic medical augmented reality control computer 3200, the data acquisition circuit 3238 is also coupled to counter timer ports 3246 and watchdog timer ports 3248. In some implementations of the holographic medical augmented reality control computer 3200, the second bridge 3216 is operably coupled to an expansion bus 3250.

In some implementations, the Ethernet® controller 3218 is operably coupled to magnetics 3252 which is operably coupled to an Ethernet® local area network 3254

With proper digital amplifiers and analog signal conditioners, the holographic medical augmented reality control computer 3200 can be programmed to drive system 110 and apparatus 220, 400, 600, 1000 and 1200, either in a predetermined sequence, or interactively modify.

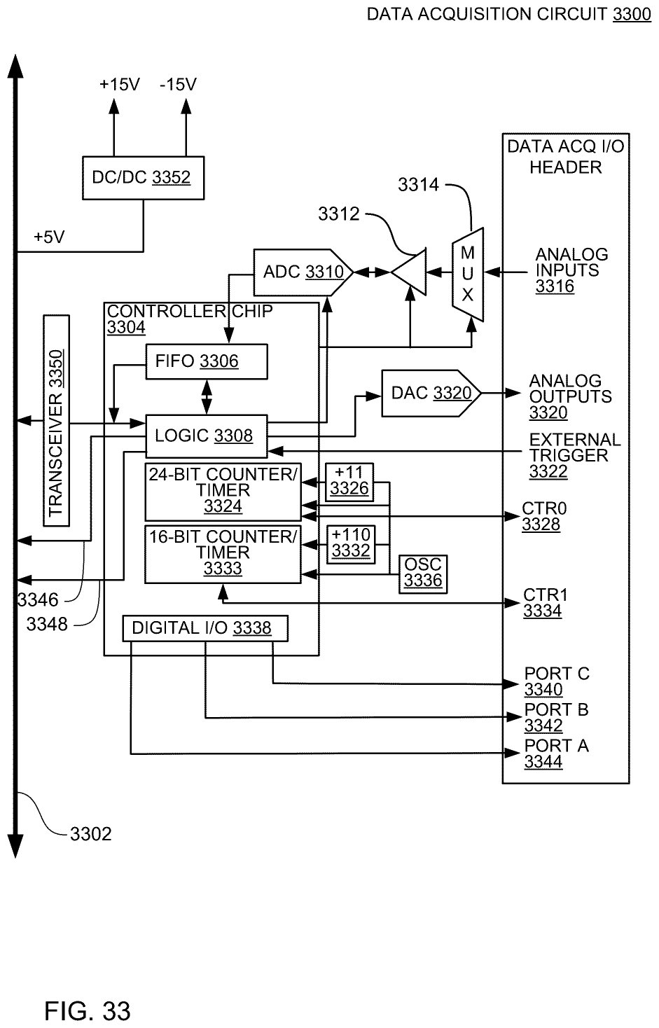

FIG. 33 is a block diagram of a data acquisition circuit 3300 of a holographic medical augmented reality control computer, according to an implementation. The data acquisition circuit 3300 is one example of the data acquisition circuit 3238 in FIG. 32 above. Some implementations of the data acquisition circuit 3300 provide 16-bit A/D performance with input voltage capability up to +/−10V, and programmable input ranges.

The data acquisition circuit 3300 can include a bus 3302, such as a conventional PC/104 bus. The data acquisition circuit 3300 can be operably coupled to a controller chip 3304. Some implementations of the controller chip 3304 include an analog/digital first-in/first-out (FIFO) buffer 3306 that is operably coupled to controller logic 3308. In some implementations of the data acquisition circuit 3300, the FIFO 3306 receives signal data from and analog/digital converter (ADC) 3310, which exchanges signal data with a programmable gain amplifier 3312, which receives data from a multiplexer 3314, which receives signal data from analog inputs 3316.

In some implementations of the data acquisition circuit 3300, the controller logic 3308 sends signal data to the ADC 3310 and a digital/analog converter (DAC) 3318. The DAC 3318 sends signal data to analog outputs. The analog outputs, after proper amplification, can be used to perform holographic medical augmented reality control computer. In some implementations of the data acquisition circuit 3300, the controller logic 3308 receives signal data from an external trigger 3322.

In some implementations of the data acquisition circuit 3300, the controller chip 3304 includes a digital input/output (I/O) component 3338 that sends digital signal data to computer output ports.

In some implementations of the data acquisition circuit 3300, the controller logic 3308 sends signal data to the bus 3302 via a control line 3346 and an interrupt line 3348. In some implementations of the data acquisition circuit 3300, the controller logic 3308 exchanges signal data to the bus 3302 via a transceiver 3350.

Some implementations of the data acquisition circuit 3300 include 12-bit D/A channels, programmable digital I/O lines, and programmable counter/timers. Analog circuitry can be placed away from the high-speed digital logic to ensure low-noise performance for important applications. Some implementations of the data acquisition circuit 3300 are fully supported by operating systems that can include, but are not limited to, DOS™, Linux™, RTLinux™, QNX™, Windows 98/NT/2200/XP/CE™, Forth™, and VxWorks™ to simplify application development.

FIG. 34 is a block diagram of a hardware and operating environment 3400 in which different implementations can be practiced. The description of FIG. 34 provides an overview of computer hardware and a suitable computing environment in conjunction with which some implementations can be implemented. Implementations are described in terms of a computer executing computer-executable instructions. However, some implementations can be implemented entirely in computer hardware in which the computer-executable instructions are implemented in read-only memory. Some implementations can also be implemented in client/server computing environments where remote devices that perform tasks are linked through a communications network. Program modules can be located in both local and remote memory storage devices in a distributed computing environment.

Computer 3402 includes a processing unit 3404, commercially available from Intel, Motorola, Cyrix and others. The computer 3402 is one implementation of system 110 in FIG. 1. The computer 3402 also includes system memory 3406 that includes random-access memory RAM 3408 and read-only memory ROM 3410. The computer 3402 also includes one or more mass storage devices 3412; and a system bus 3414 that operatively couples various system components to the processing unit 3404. The RAM 3408 and ROM 3410, and mass storage devices 3412, are types of computer-accessible media. Mass storage devices 3412 are more specifically types of nonvolatile computer-accessible media and can include one or more hard disk drives, floppy disk drives, optical disk drives, and tape cartridge drives. The processing unit 3404 executes computer programs stored on the computer-accessible media.

Computer 3402 can be communicatively connected to the Internet 3416 via a communication device, such as modem 3418. Internet 3416 connectivity is well known within the art. In one implementation, the modem 3418 responds to communication drivers to connect to the Internet 3416 via what is known in the art as a “dial-up connection.” In another implementation, the communication device is an Ethernet® or network adapter 3420 connected to a local-area network (LAN) 3422 that itself is connected to the Internet 3416 via what is known in the art as a “direct connection” (e.g., T1 line, etc.).

A user enters commands and information into the computer 3402 through input devices such as a keyboard (not shown) or a pointing device (not shown). The keyboard permits entry of textual information into computer 3402, as known within the art, and implementations are not limited to any particular type of keyboard. Pointing device permits the control of the screen pointer provided by a graphical user interface (GUI) of operating systems such as versions of Microsoft Windows®. Implementations are not limited to any particular pointing device. Such pointing devices include mice, touch pads, trackballs, remote controls and point sticks. Other input devices (not shown) can include a microphone, joystick, game pad, satellite dish, scanner, or the like.

In some implementations, computer 3402 is operatively coupled to a display device 3424. Display device 3424 is connected to the system bus 3414 through a video adapter 3426. Display device 3424 permits the display of information, including computer, video and other information, for viewing by a user of the computer. Implementations are not limited to any particular display device 3424. Such display devices include cathode ray tube (CRT) displays (monitors), as well as flat panel displays such as liquid crystal displays (LCD's). In addition to a monitor, computers typically include other peripheral input/output devices such as printers (not shown). Speakers (not shown) provide audio output of signals. Speakers are also connected to the system bus 3414.

Computer 3402 can be operated using at least one operating system to provide a graphical user interface (GUI) including a user-controllable pointer. Computer 3402 can have at least one web browser application program executing within at least one operating system, to permit users of computer 3402 to access intranet or Internet world-wide-web pages as addressed by Universal Resource Locator (URL) addresses. Examples of browser application programs include Netscape Navigator® and Microsoft Internet Explorer®.

The computer 3402 can operate in a networked environment using logical connections to one or more remote computers, such as remote computer 3428. These logical connections are achieved by a communication device coupled to, or a part of, the computer 3402. Implementations are not limited to a particular type of communications device. The remote computer 3428 can be another computer, a server, a router, a network PC, a client, a peer device or other common network node. The logical connections depicted in FIG. 34 include the local-area network (LAN) 3422 and a wide-area network (WAN). Such networking environments are commonplace in offices, enterprise-wide computer networks, intranets and the Internet.

When used in a LAN-networking environment, the computer 3402 and remote computer 3428 are connected to the local network 3422 through network interfaces or adapters 3420, which is one type of communications device 3418. When used in a conventional WAN-networking environment, the computer 3402 and remote computer 3428 communicate with a WAN through modems. The modems, which can be internal or external, is connected to the system bus 3414. In a networked environment, program modules depicted relative to the computer 3402, or portions thereof, can be stored in the remote computer 3428.

Computer 3402 also includes an operating system 3430 that can be stored on the RAM 3408 and ROM 3410, and/or mass storage device 3412, and is and executed by the processing unit 3404. Examples of operating systems include Microsoft Windows®, Apple MacOS®, Linux®, UNIX®, providing capability for supporting application programs 3432 using, for example, code modules written in the C++® computer programming language. Examples are not limited to any particular operating system, however, and the construction and use of such operating systems are well known within the art.

Instructions can be stored via the mass storage devices 3412 or system memory 3406, including one or more application programs 3432, other program modules 3434 and program data 3436.

Computer 3402 also includes power supply. Each power supply can be a battery.

Some implementations include computer instructions to perform holographic medical augmented reality control computer that can be implemented in instructions or the instructions stored via the mass storage devices 3412 or system memory 3406 in FIG. 34.

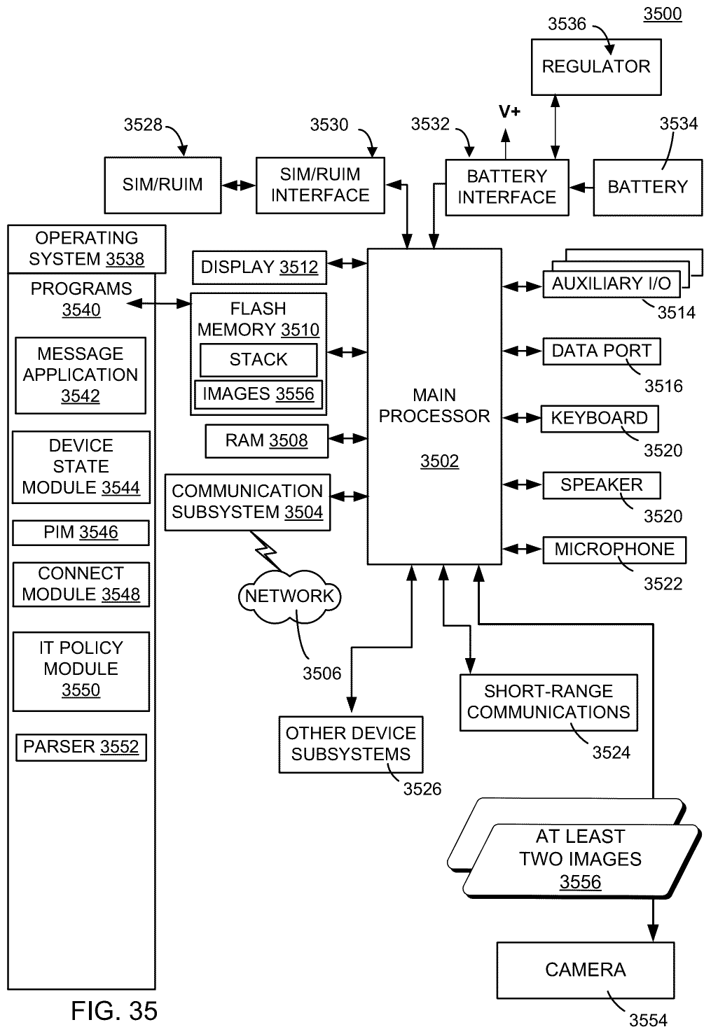

FIG. 35 is a block diagram of a medical holographic medical augmented reality control mobile device 3500, according to an implementation. The holographic medical augmented reality control mobile device 3500 includes a number of components such as a main processor 3502 that controls the overall operation of the holographic medical augmented reality control mobile device 3500. Communication functions, including data and voice communications, are performed through a communication subsystem 3504. The communication subsystem 3504 receives messages from and sends messages to a wireless network 3506. In this exemplary implementation of the holographic medical augmented reality control mobile device 3500, the communication subsystem 3504 is configured in accordance with the Global System for Mobile Communication (GSM), General Packet Radio Services (GPRS) standards, 3G, 4G, 5G and/or 6G. It will also be understood by persons skilled in the art that the implementations described herein are intended to use any other suitable standards that are developed in the future. The wireless link connecting the communication subsystem 3504 with the wireless network 3506 represents one or more different Radio Frequency (RF) channels, operating according to defined protocols specified for 4G or 5G communications. With newer network protocols, these channels are capable of supporting both circuit switched voice communications and packet switched data communications.

Although the wireless network 3506 associated with holographic medical augmented reality control mobile device 3500 is a GSM/GPRS, 3G, 4G, 5G and/or 6G wireless network in one exemplary implementation, other wireless networks may also be associated with the holographic medical augmented reality control mobile device 3500 in variant implementations. The different types of wireless networks that may be employed include, for example, data-centric wireless networks, voice-centric wireless networks, and dual-mode networks that can support both voice and data communications over the same physical base stations. Combined dual-mode networks include, but are not limited to, Code Division Multiple Access (CDMA) or CDMA2000 networks, GSM/GPRS networks, 3G, 4G, 5G and/or 6G. Some other examples of data-centric networks include WiFi 802.11, Mobitex™ and DataTAC™ network communication systems. Examples of other voice-centric data networks include Personal Communication Systems (PCS) networks like GSM and Time Division Multiple Access (TDMA) systems.

The main processor 3502 also interacts with additional subsystems such as a Random Access Memory (RAM) 3508, a flash memory 3510, a display 3512, an auxiliary input/output (I/O) subsystem 3514, a data port 3516, a keyboard 3518, a speaker 3520, a microphone 3522, short-range communications 3524 and other device subsystems 3526.

Some of the subsystems of the holographic medical augmented reality control mobile device 3500 perform communication-related functions, whereas other subsystems may provide “resident” or on-device functions. By way of example, the display 3512 and the keyboard 3518 may be used for both communication-related functions, such as entering a text message for transmission over the wireless network 3506, and device-resident functions such as a calculator or task list.

The holographic medical augmented reality control mobile device 3500 can send and receive communication signals over the wireless network 3506 after required network registration or activation procedures have been completed. Network access is associated with a subscriber or user of the holographic medical augmented reality control mobile device 3500. To identify a subscriber, the holographic medical augmented reality control mobile device 3500 requires a SIM/RUIM card 3528 (i.e. Subscriber Identity Module or a Removable User Identity Module) to be inserted into a SIM/RUIM interface 3530 in order to communicate with a network. The SIM card or RUIM 3528 is one type of a conventional “smart card” that can be used to identify a subscriber of the holographic medical augmented reality control mobile device 3500 and to customize the holographic medical augmented reality control mobile device 3500, among other aspects. Without the SIM card 3528, the holographic medical augmented reality control mobile device 3500 is not fully operational for communication with the wireless network 3506. By inserting the SIM card/RUIM 3528 into the SIM/RUIM interface 3530, a subscriber can access all subscribed services. Services may include: web browsing and messaging such as e-mail, voice mail, Short Message Service (SMS), and Multimedia Messaging Services (MMS). More advanced services may include: point of sale, field service and sales force automation. The SIM card/RUIM 3528 includes a processor and memory for storing information. Once the SIM card/RUIM 3528 is inserted into the SIM/RUIM interface 3530, it is coupled to the main processor 3502. In order to identify the subscriber, the SIM card/RUIM 352 can include some user parameters such as an International Mobile Subscriber Identity (IMSI). An advantage of using the SIM card/RUIM 3528 is that a subscriber is not necessarily bound by any single physical mobile device. The SIM card/RUIM 3528 may store additional subscriber information for a mobile device as well, including datebook (or calendar) information and recent call information. Alternatively, user identification information can also be programmed into the flash memory 3510.

The holographic medical augmented reality control mobile device 3500 is a battery-powered device and includes a battery interface 3532 for receiving one or more rechargeable batteries 3534. In one or more implementations, the battery 3534 can be a smart battery with an embedded microprocessor. The battery interface 3532 is coupled to a regulator 3536, which assists the battery 3534 in providing power V+ to the holographic medical augmented reality control mobile device 3500. Although current technology makes use of a battery, future technologies such as micro fuel cells may provide the power to the holographic medical augmented reality control mobile device 3500.

The holographic medical augmented reality control mobile device 3500 also includes an operating system 3538 and software components 3540 to 3552 which are described in more detail below. The operating system 3538 and the software components 3540 to 3552 that are executed by the main processor 3502 are typically stored in a persistent store such as the flash memory 3510, which may alternatively be a read-only memory (ROM) or similar storage element (not shown). Those skilled in the art will appreciate that portions of the operating system 3538 and the software components 3540 to 3552, such as specific device applications, or parts thereof, may be temporarily loaded into a volatile store such as the RAM 3508. Other software components can also be included.

The subset of software components 3540 that control basic device operations, including data and voice communication applications, will normally be installed on the holographic medical augmented reality control mobile device 3500 during its manufacture. Other software applications include a message application 3542 that can be any suitable software program that allows a user of the holographic medical augmented reality control mobile device 3500 to send and receive electronic messages. Various alternatives exist for the message application 3542 as is well known to those skilled in the art. Messages that have been sent or received by the user are typically stored in the flash memory 3510 of the holographic medical augmented reality control mobile device 3500 or some other suitable storage element in the holographic medical augmented reality control mobile device 3500. In one or more implementations, some of the sent and received messages may be stored remotely from the holographic medical augmented reality control mobile device 3500 such as in a data store of an associated host system with which the holographic medical augmented reality control mobile device 3500 communicates.

The software applications can further include a device state module 3544, a Personal Information Manager (PIM) 3546, and other suitable modules (not shown). The device state module 3544 provides persistence, i.e. the device state module 3545 ensures that important device data is stored in persistent memory, such as the flash memory 3510, so that the data is not lost when the holographic medical augmented reality control mobile device 3500 is turned off or loses power.

The PIM 3546 includes functionality for organizing and managing data items of interest to the user, such as, but not limited to, e-mail, contacts, calendar events, voice mails, appointments, and task items. A PIM application has the ability to send and receive data items via the wireless network 3506. PIM data items may be seamlessly integrated, synchronized, and updated via the wireless network 3506 with the mobile device subscriber's corresponding data items stored and/or associated with a host computer system. This functionality creates a mirrored host computer on the holographic medical augmented reality control mobile device 3500 with respect to such items. This can be particularly advantageous when the host computer system is the mobile device subscriber's office computer system.

The holographic medical augmented reality control mobile device 3500 also includes a connect module 3548, and an IT policy module 3550. The connect module 3548 implements the communication protocols that are required for the holographic medical augmented reality control mobile device 3500 to communicate with the wireless infrastructure and any host system, such as an enterprise system, with which the holographic medical augmented reality control mobile device 3500 is authorized to interface.

The connect module 3548 includes a set of APIs that can be integrated with the holographic medical augmented reality control mobile device 3500 to allow the holographic medical augmented reality control mobile device 3500 to use any number of services associated with the enterprise system. The connect module 3548 allows the holographic medical augmented reality control mobile device 3500 to establish an end-to-end secure, authenticated communication pipe with the host system. A subset of applications for which access is provided by the connect module 3548 can be used to pass IT policy commands from the host system to the holographic medical augmented reality control mobile device 3500. This can be done in a wireless or wired manner. These instructions can then be passed to the IT policy module 3550 to modify the configuration of the holographic medical augmented reality control mobile device 3500. Alternatively, in some cases, the IT policy update can also be done over a wired connection.

The IT policy module 3550 receives IT policy data that encodes the IT policy. The IT policy module 3550 then ensures that the IT policy data is authenticated by the holographic medical augmented reality control mobile device 3500. The IT policy data can then be stored in the flash memory 3510 in its native form. After the IT policy data is stored, a global notification can be sent by the IT policy module 3550 to all of the applications residing on the holographic medical augmented reality control mobile device 3500. Applications for which the IT policy may be applicable then respond by reading the IT policy data to look for IT policy rules that are applicable.

The IT policy module 3550 can include a parser 3552, which can be used by the applications to read the IT policy rules. In some cases, another module or application can provide the parser. Grouped IT policy rules, described in more detail below, are retrieved as byte streams, which are then sent (recursively) into the parser to determine the values of each IT policy rule defined within the grouped IT policy rule. In one or more implementations, the IT policy module 3550 can determine which applications are affected by the IT policy data and send a notification to only those applications. In either of these cases, for applications that are not being executed by the main processor 3502 at the time of the notification, the applications can call the parser or the IT policy module 3550 when they are executed to determine if there are any relevant IT policy rules in the newly received IT policy data.

All applications that support rules in the IT Policy are coded to know the type of data to expect. For example, the value that is set for the “WEP User Name” IT policy rule is known to be a string; therefore the value in the IT policy data that corresponds to this rule is interpreted as a string. As another example, the setting for the “Set Maximum Password Attempts” IT policy rule is known to be an integer, and therefore the value in the IT policy data that corresponds to this rule is interpreted as such.

After the IT policy rules have been applied to the applicable applications or configuration files, the IT policy module 3550 sends an acknowledgement back to the host system to indicate that the IT policy data was received and successfully applied.

Other types of software applications can also be installed on the holographic medical augmented reality control mobile device 3500. These software applications can be third party applications, which are added after the manufacture of the holographic medical augmented reality control mobile device 3500. Examples of third party applications include games, calculators, utilities, etc.

The additional applications can be loaded onto the holographic medical augmented reality control mobile device 3500 through at least one of the wireless network 3506, the auxiliary I/O subsystem 3514, the data port 3516, the short-range communications subsystem 3524, or any other suitable device subsystem 3524. This flexibility in application installation increases the functionality of the holographic medical augmented reality control mobile device 3500 and may provide enhanced on-device functions, communication-related functions, or both. For example, secure communication applications may enable electronic commerce functions and other such financial transactions to be performed using the holographic medical augmented reality control mobile device 3500.

The data port 3516 enables a subscriber to set preferences through an external device or software application and extends the capabilities of the holographic medical augmented reality control mobile device 3500 by providing for information or software downloads to the holographic medical augmented reality control mobile device 3500 other than through a wireless communication network. The alternate download path may, for example, be used to load an encryption key onto the holographic medical augmented reality control mobile device 3500 through a direct and thus reliable and trusted connection to provide secure device communication.

The data port 3516 can be any suitable port that enables data communication between the holographic medical augmented reality control mobile device 3500 and another computing device. The data port 3516 can be a serial or a parallel port. In some instances, the data port 3516 can be a USB port that includes data lines for data transfer and a supply line that can provide a charging current to charge the battery 3534 of the holographic medical augmented reality control mobile device 3500.

The short-range communications subsystem 3524 provides for communication between the holographic medical augmented reality control mobile device 3500 and different systems or devices, without the use of the wireless network 3506. For example, the subsystem 3524 may include an infrared device and associated circuits and components for short-range communication. Examples of short-range communication standards include standards developed by the Infrared Data Association (IrDA), Bluetooth, and the 802.11 family of standards developed by IEEE.

In use, a received signal such as a text message, an e-mail message, or web page download will be processed by the communication subsystem 3504 and input to the main processor 3502. The main processor 3502 will then process the received signal for output to the display 3512 or alternatively to the auxiliary I/O subsystem 3514. A subscriber may also compose data items, such as e-mail messages, for example, using the keyboard 3518 in conjunction with the display 3512 and possibly the auxiliary I/O subsystem 3514. The auxiliary subsystem 3514 may include devices such as: a touch screen, mouse, track ball, infrared fingerprint detector, or a roller wheel with dynamic button pressing capability. The keyboard 3518 is preferably an alphanumeric keyboard and/or telephone-type keypad. However, other types of keyboards may also be used. A composed item may be transmitted over the wireless network 3506 through the communication subsystem 3504.

For voice communications, the overall operation of the holographic medical augmented reality control mobile device 3500 is substantially similar, except that the received signals are output to the speaker 3520, and signals for transmission are generated by the microphone 3522. Alternative voice or audio I/O subsystems, such as a voice message recording subsystem, can also be implemented on the holographic medical augmented reality control mobile device 3500. Although voice or audio signal output is accomplished primarily through the speaker 3520, the display 3512 can also be used to provide additional information such as the identity of a calling party, duration of a voice call, or other voice call related information.

In some implementations, the holographic medical augmented reality control mobile device 3500 includes a camera 3554 receiving a plurality of images 3556 from and examining pixel-values of the plurality of images 3556.

### CONCLUSION

A system for points-based registration of a medical image to a patient in holographic medical augmented reality is described. A technical effect of the points-based registration of a medical image to a patient in holographic medical augmented reality is a medical hologram is registered on a surgical site. Although specific implementations are illustrated and described herein, it will be appreciated by those of ordinary skill in the art that any arrangement which is calculated to achieve the same purpose may be substituted for the specific implementations shown. This application is intended to cover any adaptations or variations. For example, although some parts of this disclosure are described in procedural terms, one of ordinary skill in the art will appreciate that implementations can be made in object-oriented or any other architecture that provides the required function.

In particular, one of skill in the art will readily appreciate that the names of the methods and apparatus are not intended to limit implementations. Furthermore, additional methods and apparatus can be added to the components, functions can be rearranged among the components, and new components to correspond to future enhancements and physical devices used in implementations can be introduced without departing from the scope of implementations. One of skill in the art will readily recognize that implementations are applicable to future medical images, different user interfaces and command interfaces.

The terminology used in this application meant to include all medical images and alternate technologies which provide the same functionality as described herein.

1. A method of points-based registration of a medical hologram to a surgical site of a patient, the method comprising:
receiving created registration points on the medical hologram, the medical hologram being stored in a memory;
receiving placed registration points on the surgical site of the patient, wherein the medical hologram is stored in the memory; and
registering the medical hologram on the surgical site of the patient in reference to the placed registration points.

2. The method of claim 1 wherein the receiving the created registration points on the medical hologram further comprises:
projecting the medical hologram of a point on top of a finger position, in the memory;
receiving a command in the memory to register the finger position as one of the registration points;
registering the finger position as one of the registration points in the memory;
determining whether or not an exit command has been received; and
tracking the finger position and reiterating receiving the created registration points if no exit command has been received.

3. The method of claim 1 wherein the receiving of the placed registration points on a medical image of the patient further comprises:
displaying a registered point on the medical hologram;
tracking a finger position;
projecting the registered point of the medical hologram on the finger position;
receiving a command to place the registered point of the medical hologram on the finger position on a landmark of the surgical site;
placing the registered point of the medical hologram on the finger position as one of the registration points on the landmark of the surgical site;
determining whether another created registration point exists on the medical hologram; and
tracking the finger position and reiterating the receiving the placed registration points on the medical image of the patient if no exit command has been received.

4. The method of claim 2 wherein the registering the finger position as the registration point in the memory further comprises:
generating an indicator of placed registration points on the surgical site;

5. The method of claim 1 further comprising before the receiving the created registration points:
receiving an indication of a selection of the medical hologram of the patient in a medical image stored in the memory; and
receiving an indication of a selection of registration or placement.

6. The method of claim 5 wherein the receiving the indication of the selection of the medical hologram of the patient further comprises:
displaying the medical hologram of the patient; and
receiving an indication of a selected medical hologram of the patient.

7. The method of claim 5 wherein the receiving the indication of the selection of registration or placement further comprises:
displaying actions on a selected medical hologram of the patient;
receiving an indicator of a selected action of placement; and
scaling the selected medical hologram of the patient.

8. An apparatus comprising:
a creator of registration points on a medical hologram which is operable to generate an indicator of created registration points;
a placer of registration points on a surgical site, the placer being operably coupled to the creator, the placer being operable to generate an indicator of placed registration points on the surgical site from the indicator of created registration points; and
a registrar of the medical hologram on the surgical site, the registrar being operable to generate an indicator of the placed registration points on the surgical site from the indicator of placed registration points, the registrar being operably coupled to the placer.

9. The apparatus of claim 8 further comprising:
a selector of the medical hologram of a patient in a medical image, the selector being operable to generate an indicator of a selected medical hologram of the patient; and
a registration selector that is operable to receive the indicator and generate an indicator of placement, the registration selector being is operably coupled to the selector of the medical hologram of the patient and operably coupled to the creator of registration points on the medical hologram.

10. The apparatus of claim 8 wherein the creator of registration points further comprises:
a recognizer of a finger position that is operable to recognize a user's finger;
a tracker of the finger position, that is operable to track the finger position from the user's finger;
a projector that is operable to project the medical hologram of a point on top of the finger position;
a receiver of a command to register the finger position as a registration point;
a registrar of the finger position as a registration point; and
an evaluator of a next command that is operable to determine that the command is not an exit command to continue processing the finger position by the tracker at and registering the finger position as a registration point by the registrar.

11. The apparatus of claim 8 wherein the placer further comprises:
a displayer of a next created registration point on the medical hologram;
a finger position tracker;
a projector of a created registration point of the medical hologram a finger position;
a receiver of a command to place the created registration point of the medical hologram on the finger position on a landmark of the surgical site;
a placer of the next created registration point of the medical hologram on the finger position as a registration point on the landmark of the surgical site;
an evaluator of a registered point on the medical hologram that is operable when the registered point exists on the medical hologram to continue processing the registered point on the medical hologram and placing the registered point of the medical hologram on the finger position as a registration point on the landmark of the surgical site.

12. The apparatus of claim 8 wherein the registrar further comprises:
a generator of an indicator of placed registration points on the surgical site.

13. An apparatus comprising:
a microprocessor that has program instructions that are adapted to register a medical image to a medical hologram of a patient;
wherein the program instructions of the microprocessor are further adapted to:
receive created registration points on the medical hologram, the medical hologram being stored in a memory;
receive placed registration points on the medical image of the patient, wherein the medical image is stored in the memory; and
register the medical hologram on the medical image of the patient.

14. The apparatus of claim 13 wherein the program instructions of the microprocessor to receive the created registration points on the medical hologram are further adapted to:
project the medical hologram of a point on top of a finger position, in the memory;
receive a command in the memory to register the finger position as a registration point;
register the finger position as a registration point in the memory;
determine whether or not an exit command has been received; and
track the finger position and reiterate receive the created registration points if no exit command has been received.

15. The apparatus of claim 13 wherein the program instructions of the microprocessor to receive the placed registration points on the medical image of the patient are further adapted to:
display a registered point on the medical hologram;
track a finger position;
project the registered point of the medical hologram on the finger position;
receive a command to place the registered point of the medical hologram on the finger position on a landmark of a surgical site;
place the registered point of the medical hologram on the finger position as a registration point on the landmark of the surgical site;
determine whether another created registration point exists on the medical hologram; and
track the finger position and reiterate project the registered point of the medical hologram on the finger position if no exit command has been received.

16. The apparatus of claim 13 wherein the program instructions of the microprocessor to register the medical hologram on the medical image of the patient are further adapted:
generate an indicator of placed registration points on a surgical site;

17. The apparatus of claim 13 wherein the program instructions of the microprocessor to are further adapted:
receive an indication of a selection of the medical hologram of the patient in the medical image stored in the memory; and
receive an indication of a selection of registration.

18. The apparatus of claim 17 wherein the program instructions of the microprocessor to receive the indication of the selection of the medical hologram of the patient are further adapted to:
display the medical hologram of the patient; and
receive an indicator of a selected medical hologram of the patient.

19. The apparatus of claim 17 wherein the program instructions of the microprocessor to receive the indication of the selection of registration are further adapted:
display actions on a selected medical hologram of the patient;
receive an indicator of a selected action of placement; and
scale the selected medical hologram of the patient.
